(19) United States
(12) Patent Application Publication
(10) Pub. No.: US 2010/0173134 A1
(43) Pub. Date: Jul. 8, 2010

(54) Film and Device Using Layer Based on Ribtan Material

(75) Inventors: Pavel Khokhlov, Moscow (RU); Pavel I. Lazarev, London (GB)

Correspondence Address: HOUST CONSULTING, P.O. BOX 2688, SARATOGA, CA, 95070-0688, US

(73) Assignee: CARBEN SEMICON LIMITED, Nicosia (CY)

(21) Appl. No.: 12/492,127

(22) Filed: Jun. 25, 2009

Related U.S. Application Data
(60) Provisional application No. 61/076,091, filed on Jun. 26, 2008.

(51) Int. Cl. B32B 1/00; C07D 471/22; B32B 9/04; B32B 15/04; B32B 17/06; B32B 27/06; B05D 5/06; C07D 471/06; C07C 49/603

(52) U.S. Cl. 428/174; 546/27; 428/446; 428/704; 428/409; 428/426; 428/457; 428/412; 428/480; 428/473.5; 427/66; 546/37; 568/326

(57) ABSTRACT

The present invention relates generally to the field of electronics. More specifically, the present invention relates to film and device using layer based on carbon-based ribtan material. According to present invention, the film comprises at least one optically transparent and electrically conductive layer based on a ribtan material.

### CROSS REFERENCE TO RELATED APPLICATIONS

This application is related to the following applications that are filed concurrently herewith.

United States Provisional Patent Application entitled “Patterned Integrated Circuit and Method of Production Thereof,” Attorney Docket 7006-0351; and

United States Provisional Patent Application entitled “Integrated Circuit with Ribtan Interconnects,” Attorney Docket 7006-0301.

The disclosures of both of the above applications are incorporated herein by reference in their entirety.

### FIELD OF THE INVENTION

The present invention relates generally to the field of electronics. More specifically, the present invention relates to film and device using layer based on carbon-based ribtan material.

### BACKGROUND OF THE INVENTION

Many modern and/or emerging applications require films and at least one device electrode that have not only high electrical conductivity, but high optical transparency as well. Such applications include, but are not limited to flexible displays (e.g., electro-phoretics, electro-luminescence, electrochromatic), touch screens (e.g., analog, resistive, improved analog, X/Y matrix, capacitive), rigid displays (e.g., liquid crystal (LCD), plasma (PDP), light emitting diode (LED), organic light emitting diode (OLED)), solar cells and microfluidics (e.g. electrowetting on dielectric (EWOD)). As used herein, a layer of material or a sequence of several layers of different materials is said to be “transparent” when the layer or layers permit at least 50% of the ambient electromagnetic radiation in relevant wavelengths to be transmitted through the layer or layers.

Currently, the most common transparent electrodes are transparent conducting oxides (TCOs), specifically indium-tin-oxide (ITO) on glass. However, ITO can be an inadequate solution for many of the above-mentioned applications (e.g., due to its relatively brittle nature and correspondingly inferior flexibility and abrasion resistance), and the indium component of ITO is rapidly becoming a scarce commodity. Hence, more robust and abundant transparent conductor materials are being explored.

Indium tin oxide (ITO) and fluorine tin oxide (FTO) have been widely used as window electrodes in optoelectronic devices. The use of metal oxides, however, appear to be increasingly problematic due to (i) the limited availability of the element indium on earth, (ii) their instability in the presence of acid or base, (iii) their susceptibility to ion diffusion into polymer layers, and (iv) the current leakage of FTO devices caused by FTO structure defects.

The search for novel electrode materials with good stability, high transparency and excellent conductivity is therefore a crucial goal for optoelectronics. Graphene, two-dimensional lattice of graphite, exhibits remarkable electronic properties that qualify it for applications in future optoelectronic devices. Recently, transparent and conductive graphene-based composites have been prepared by incorporation of graphene sheets into polystyrene or silica. However, the conductivity of such transparent composites is low, typically ranging from 10⁻³ to 1 S/cm depending upon the graphene sheet loading level, which makes the composites incapable of serving as window electrodes in optoelectronic devices. Transparent, conductive graphene electrodes for dye-sensitized solar cells were studied by Xuan Wang, Linjie Zhi, and Klaus Mullen and published in Nano Letters, vol. 8, no. 1 (2008), pp 323-327. Herein, the authors present a simple approach for the fabrication of conductive, transparent, and ultrathin graphene films from exfoliated graphite oxide, followed by thermal reduction. The obtained graphene films with a thickness of approximately 10 nm exhibit a high conductivity of 550 S/cm, which is comparable to that of polycrystalline graphite (1250 S/cm), and a transparency of more than 70% over 1000-3000 nm. The application of graphene films as window electrodes in solid-state dye sensitized solar cells is demonstrated. Graphene sheets have been produced either by mechanical exfoliation via repeated peeling of highly ordered pyrolytic graphite (HOPG) or by chemical oxidation of graphite. Considering the facile solution processing, the oxidation of graphite was preferred for this study. Oxygen-containing functional groups render the graphite oxide (GO) hydrophilic and dispersible in water. GO was produced through acid oxidation of flake graphite. The primary product was suspended in water under ultrasonication for half an hour, followed by centrifuged at 4000 rpm for 30 min. The obtained supernate was dried via evaporation of water under vacuum. Then, the solid was dispersed again in water (1.5 mg/mL) by ultrasonication for 2 h and centrifuged at 10 000 rpm for 15 min to further remove aggregates. Finally, the supernate was collected and ready for use. Such aqueous dispersion of exfoliated GO could stay stable for several weeks, free of any obvious precipitates. The exfoliated graphene sheets with lateral dimensions of several tens to hundreds of nanometers were observed under scanning electron microscopy (SEM). However, the obtained GO is electrically insulating due to the heavy oxygenation of graphene sheets. Reduction of GO, either by chemical reaction using reducing agent, such as NaBH₄ or dimethylhydrazine, or by pyrolysis at high temperatures, has been reported to render the material electrically conductive. In order to avoid agglomeration of graphene sheets after reduction, other host molecules such as polymers must be used, which hamper the electron-transfer property of graphene. As it was described in cited paper, the GO sheets were deposited on the surface of the substrate and then reduced into graphene.

Graphene-silica composite thin films as transparent conductors were studied by Supinda Watcharotone, Dmitriy A. Dikin, et al. and published in Nano Letters, (2007), vol. 7(7), pp. 1888-1892. Transparent and electrically conductive composite silica films were fabricated on glass and hydrophilic SiOx/silicon substrates by incorporation of individual graphene oxide sheets into silica sols followed by spin-coating, chemical reduction, and thermal curing. The resulting films were characterized by SEM, AFM, TEM, low-angle X-ray reflectivity, XPS, UV-vis spectroscopy, and electrical conductivity measurements. The electrical conductivity of the films compared favorably to those of composite thin films of carbon nanotubes in silica.

Carbon nanotube films for transparent and plastic electronics were studied by Gruner G. in J. Mater. Chem., 2006, vol. 16, pp. 3533-3539. A two-dimensional network—often referred to as a thin film—of carbon nanotubes can be regarded as a novel transparent electronic material with excellent—and tunable—electrical, optical and mechanical properties. The films display high conductivity, high carrier mobility and optical transparency, in addition to flexibility, robustness and environmental resistance. These attributes, coupled with room temperature printing or spraying technology, ensure that the material will have a significant impact on a variety of emerging technologies and markets, ranging from macro-electronics to solid state lighting, organic solar cells and smart fabrics. The performance parameters of the first devices fabricated—smart windows, OLEDs and organic solar cells—indicate that the material is ready for product development.

Properties and characterization of carbon-nanotube-based transparent conductive coating were studied by Trottier, C. M., Glatkowski, P. et al. in Journal of the Society for Information Display (2005), vol. 13(9), pp. 759-763. Transparent and electrically conductive coatings and films have a variety of fast-growing applications ranging from window glass to flat-panel displays. These mainly include semiconductive metal oxides such as indium tin oxide (ITO) and polymers such as poly(3,4-ethylenedioxythiophene) doped and stabilized with poly(styrenesulfonate) (PEDOT/PSS). In cited paper, the authors show alternatives to ITO and conducting polymers, using single-wall carbon nanotubes (SWNT). This paper reports on optoelectronic properties and structure characterization of these materials.

### SUMMARY OF THE INVENTION

The present invention provides a film comprising at least one optically transparent and electrically conductive layer based on a ribtan material.

In a further aspect, the present invention provides a device comprising at least one optically transparent and electrically conductive layer based on a ribtan material.

In a yet further aspect, the present invention provides a method of producing at least one ribtan layer on a substrate, which comprises the following steps: (a) application of a solution of at least one π-conjugated organic compound of the general structural formula I or a combination of the organic compounds of the general structural formula I on a substrate:

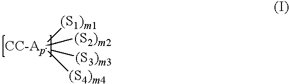

where CC is a predominantly planar carbon-conjugated core; A is a hetero-atomic group; p is 0, 1, 2, 3, 4, 5, 6, 7, or 8; S₁, S₂, S₃, and S₄ are substituents, m1, m2, m3 and m4 are 0, 1, 2, 3, 4, 5, 6, 7, or 8; and sum (m1+m2+m3+m4) is 1, 2, 3, 4, 5, 6, 7, 8, 9 or 10; (b) drying with formation of a solid precursor layer, and (c) formation of a ribtan layer. Said formation step (c) is characterized by a level of vacuum, a composition and pressure of an ambient gas, and a time dependence of temperature which are selected so as to ensure a creation of predominantly planar graphene-like structures in the ribtan layer. At least one said graphene-like structure possesses conductivity and is predominantly continuous within the entire ribtan layer, and wherein thickness of the ribtan layer is in the range from approximately 1 nm to 1000 nm.

In still further aspect, the present invention provides a method of producing a ribtan layer on a substrate, which comprises the following steps: (a) preparation of a solution of one π-conjugated organic compound of a general structural formula II or a combination of the organic compounds of the general structural formula II capable of forming supramolecules:

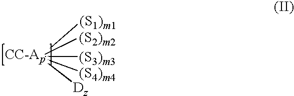

where CC is a predominantly planar carbon-conjugated core; A is an hetero-atomic group; p is 0, 1, 2, 3, 4, 5, 6, 7, or 8; S₁, S₂, S₃, S₄ and D are substituents, where S₁, S₂, S₃, and S₄ are substituents providing a solubility of the organic compound in suitable solvent and D is a substituent which produces reaction centers selected from the list comprising free radicals and benzyne fragments on the predominantly planar carbon-conjugated cores after elimination this substituent during subsequent step (d); m1, m2, m3 and m4 are 0, 1, 2, 3, 4, 5, 6, 7, or 8; sum (m1+m2+m3+m4) is 1, 2, 3, 4, 5, 6, 7, 8, 9 or 10, and z is 0, 1, 2, 3 or 4; (b) deposition of a layer of the solution on the substrate followed by an alignment action upon the solution in order to ensure preferred alignment of the supramolecules; (c) drying with formation of a solid precursor layer; and (d) applying an external action upon the solid precursor layer stimulating low-temperature carbonization and formation of the ribtan layer.

### BRIEF DESCRIPTION OF THE DRAWINGS

The invention is better understood from reading the following detailed description of the preferred embodiments, with reference to the accompanying figures in which:

FIG. 1 shows chemical formulas of six isomers of Bis (carboxybenzimidazoles) of Perylenetetracarboxylic acids;

FIG. 2 schematically shows the disclosed anisotropic semiconductor film after the annealing step, wherein the planes of π-conjugated organic compound are oriented predominantly perpendicularly to the substrate surface;

FIG. 3 shows the typical annealing regime;

FIG. 4 shows the results of thermo-gravimetric analysis of the bis-carboxy DBI PTCA layer;

FIG. 5 schematically shows the disclosed anisotropic semiconductor film after the pyrolysis of the organic compound, wherein the planes of carbon-conjugated residues are oriented predominantly perpendicularly to the substrate surface;

FIG. 6 schematically shows a graphene-like carbon-based structure;

FIG. 7 schematically shows an embodiment of the disclosed anisotropic semiconductor film, wherein the planes of graphene-like carbon-based structures are oriented predominantly perpendicularly to the substrate surface;

FIG. 8 shows TEM image of bis-carboxy DBIPTCA annealed at 650° C. for 30 minutes;

FIG. 9 shows electron diffraction on bis-carboxy DBIPTCA film annealed at 650° C. for 30 minutes;

FIG. 10 shows absorption spectra of the annealed and dried layer of bis-carboxy DBI PTCA;

FIG. 11 shows transmittance spectra of the annealed layer of bis-carboxy DBI PTCA ITO and FTO layers;

FIG. 12 shows Raman spectra of the annealed samples;

FIG. 13 shows resistivity as a function of maximum annealing temperature (Tₘₐₓ);

FIG. 14 shows resistivity as a function of time of sample exposure at maximum temperature;

FIG. 15 shows the voltage-current characteristics obtained at different annealing temperatures on bis-carboxy DBIPTCA layer;

FIG. 16 shows a double-layer organic photovoltaic device disclosed in present invention;

FIG. 17 shows the energy band diagram of the double-layer organic photovoltaic device; and

FIG. 18 shows current-voltage characteristics of the samples of ribtan material made of a sulfo derivate of a molecule having structures 24 shown in the Table 2;

FIG. 19 shows current-voltage characteristics of the samples of ribtan material made of a sulfo derivate of a molecule having structures 25 shown in the Table 2;

FIG. 20 shows the chemical reactions taken place at a low-temperature carbonization process according to the present invention;

FIG. 21 shows silicon solar cell with transparent ribtan electrode;

FIG. 22 shows an optical transmittance of ribtan films in UV, visible and near IR regions of optical spectrum; and

FIG. 23 shows polarizing properties of a ribtan layer.

### DETAILED DESCRIPTION OF THE INVENTION

The general description of the present invention having been made, a further understanding can be obtained by reference to the specific preferred embodiments, which are given herein only for the purpose of illustration and are not intended to limit the scope of the appended claims. In addition, an aspect described in conjunction with a particular embodiment is not necessarily limited to that embodiment and can be practiced in any other embodiments of the invention.

Definitions of various terms used in the description and claims of the present invention are listed below.

The term “visible spectral range” refers to a spectral range having the lower boundary approximately equal to 400 nm, and upper boundary approximately equal to 750 nm.

Hereinafter the name ribtan material is used for a new material disclosed. Ribtan is a carbon material which can exist in two modification: 1) it can consist of aligned graphene-like nanoribbons which are aligned parallel to each other and perpendicular (edge-on) to surface of substrate, and 2) it can consist of aligned graphene-like sheets which are aligned parallel to each other and parallel (face-on or homeotropic) to the surface of substrate. Graphene-like nanoribbons are narrow strips of graphene—one-atom-thick planar sheet of sp²-bonded carbon atoms that are densely packed in a honeycomb crystal lattice. Graphene-like sheets are wide sheets of graphene—one-atom-thick planar sheet of sp²-bonded carbon atoms that are densely packed in a honeycomb crystal lattice. The layers made of ribtan will be hereinafter named as ribtan layers. Technology of ribtan layers production will be hereinafter named ribtan technology. The ribtan technology is based on a thermally induced carbonization of organic compounds with predominantly planar carbon-conjugated cores.

Ribtan technology comprises a sequence of technological steps. The first step in ribtan technology is cascade crystallization process. Cascade crystallization is a method of the consecutive multi-step crystallization process for production of the solid precursor layers with ordered structure. The process involves a chemical modification step and several steps of ordering during the formation of the solid precursor layer. The chemical modification step introduces hydrophilic groups on the periphery of the molecule in order to impart amphiphilic properties to the molecule. Amphiphilic molecules stack together into supramolecules. The specific concentration is chosen, at which supramolecules are converted into a liquid-crystalline state to form a lyotropic liquid crystal (LLC), which is the next step of ordering. The LLC is deposited under the action of a shear force onto a substrate, so that the shear force direction determines the crystal axis direction in the resulting solid precursor layer. This shear-force—assisted directional deposition is the next step of ordering, representing the global ordering of the crystalline or polycrystalline structure on the substrate surface. The last step of the process is drying/crystallization, which converts the lyotropic liquid crystal into a solid precursor layer with highly ordered molecular structure. Planes of π-conjugated molecules in the formed precursor layer can be aligned parallel (face-on or homeotropic) or perpendicular (edge-on) to the surface of substrate depending on molecular structure and/or coating technique. Control over the precursor layer structure allows formation of layers comprising continuous graphene-like nanoribbons or graphene-like sheets with high electron mobility and low resistivity during carbonization process.

Cascade crystallization is followed by carbonization process named hereinafter as a step of formation of the metallic ribtan layer. Carbonization is the term for a set of conversion reaction of an organic substance into carbon. Carbonization is usually a heating cycle. Carbonization might be performed with a heater such as a radiating heater, resistive heater, heater using an ac-electric or magnetic field, heater using a flow of heated liquid, and heater using a flow of heated gas. Carbonization is performed in a reducing or inert atmosphere with a simultaneous slow heating, over a range of temperature that varies with the nature of the particular precursor and may extend to 2500° C. Carbonization is usually a complex process and several reactions may take place sequentially or simultaneously such as pyrolysis and fusion. Also carbonization process may be enhanced by addition of gas-phase or liquid-phase catalyst or reagents.

The first stage of carbonization is a pyrolysis process. Pyrolysis is the chemical decomposition of a condensed substance. Common products of pyrolysis are volatile compounds containing non-carbon atoms and solid carbon residue. Preferably the diffusion of the volatile compounds to the atmosphere occurs slowly to avoid disruption and rupture of the carbon network. As a result, carbonization is usually a slow process. Its duration may vary considerably depending on the composition of the end-product, type of precursor, thickness of the material, and other factors. Pyrolysis process converts the solid precursor layer into essentially all carbon (product of pyrolysis).

The second stage of carbonization is a fusion reaction. Fusion (in other words condensation or polymerization) in ribtan technology is chemical reactions between neighboring molecules or their pyrolized residues and which lead to growth of continuous graphene-like nanoribbons (in case of edge-on orientation of molecules in precursor layer) or stacked graphene-like sheets (in case of homeotropic precursor layer).

Several intermediate materials are formed during carbonization process. Product of pyrolysis consists of carbon cores separated by gaps. All structural parameters of the pyrolysis product (interplanar spacing; structure of residual carbon cores; dimensions of gaps between residual carbon cores and their concentration; orientation of carbon cores in respect to the substrate surface) are determined by structure of a precursor layer. Fusion process of product of pyrolysis leads to formation of an array of graphene-like nanoribbons or stacked graphene-like sheets with gaps. Generally, atomic structure of the nanoribbons or sheets with gaps is similar to the product of pyrolysis, but islands of sp² carbon atoms grow and get ribbon-like or sheet-like morphology. Structural parameters of the nanoribbons or sheets with gaps such as structure of residual carbon cores, dimensions of gaps between residual carbon cores and their concentration—are determined by parameters of carbonization process including but not limited to temperature, time, composition and pressure of ambient gas. Interplanar spacing and orientation of carbon cores in respect to the substrate surface depends on structure of precursor layer.

The intermediate materials described above have different electronic properties, especially conductivity. Mobility of charge carriers within graphene-like nanoribbon or graphene-like sheet reaches high values, which are approximately equal to 2*10⁵ cm²V⁻¹s⁻¹. Mobile charge carriers overcome the gaps between the graphene-like nanoribbons by hopping, and this conductivity is named hopping conductivity. Electrical properties of the intermediate material depend on the concentration of gaps in the graphene-like nanoribbons or graphene-like sheets. Larger concentration of gaps leads to a smaller total electrical conductivity of the layer. By controlling the concentration of gaps, the layers can be formed in any of three states: insulating, semiconducting and metallic. The semiconducting state and the metallic state can be characterized as electrical-conducting states. In the insulating state the material has resistivity in the range of 10⁸ Ω*cm to 10¹⁸ Ω*cm. In the semiconducting state, the resistivity of the material is in the range of 10⁻¹ Ω*cm to 10⁸ Ω*cm. In the metallic state, the resistivity of the material is in the range of 10⁻⁶ Ω*cm to 10⁻¹ Ω*cm.

There is no energy gap in the energy band structure of the graphene-like sheet. One possible method of creating an energy gap is the formation of thin graphene-like nanoribbons. The width of these graphene-like nanoribbons is selected so as to control the energy gap in electron energy distribution spectrum that is formed due to quantum-dimensional effects. Formation of the ordered graphene-like nanoribbons by fusion reaction in the ribtan structure allows precise control of a nanoribbon width simply by controlling the layer thickness. The precursor layer thickness depends only on solution concentration and coating parameters for layers obtained from LLC solution.

The ribtan technology allows the high volume production of ribtan layers over large surface (from several square millimeters to several square meters or larger).

In one preferable embodiment of the present invention, the film comprises at least one optically transparent and electrically conductive layer based on a ribtan material. In one embodiment of the present invention, the film comprises two or more optically transparent and electrically conductive layers, wherein at least two said layers are based on different ribtan materials. In one embodiment of the disclosed film, at least one optically transparent and electrically conductive layer is transparent in the UV, visible and near IR regions of optical spectrum. In another embodiment of the disclosed film, at least one optically transparent and electrically conductive layer possesses polarizing properties in the visible spectral range. In still another embodiment of the disclosed film, at least one optically transparent and electrically conductive layer has an optical transparency of at least 80% for 550 nm light and a resistivity of less than 0.002-0.029 Ohm·cm. In another embodiment of the present invention, the film further comprises a substrate. In still another embodiment of the disclosed film, the substrate is made of a flexible material. In yet another embodiment of the disclosed film, the substrate is made of a rigid material. In one embodiment of the disclosed film, the surface of the substrate is flat, convex, concave, or any combination thereof. In another embodiment of the disclosed film, the substrate is made of one or several materials of the group comprising Si, Ge, SiGe, GaAs, diamond, quartz, silicon carbide, indium arsenide, indium phosphide, silicon germanium carbide, gallium arsenic phosphide, gallium indium phosphide, plastics, glasses, ceramics, metal-ceramic composites, metals, and comprises doped regions, circuit elements, and multilevel interconnects. In still another embodiment of the disclosed film, the plastic substrate is selected from the group comprising polycarbonate, Mylar, polyethylene terephthalate (PET) and polyimide. In yet another embodiment of the disclosed film, the substrate is transparent for electromagnetic radiation in the visible spectral range. In one embodiment of the present invention, the film further comprises a transparent adhesive layer which may be made of polyvinylbutyral or polyacrylate. In another embodiment of the present invention, the film further comprises a protective coating on top of the transparent adhesive layer.

In still another embodiment of the disclosed film, the ribtan material is prepared using at least one π-conjugated organic compound of the general structural formula I or a combination of the organic compounds of the general structural formula I:

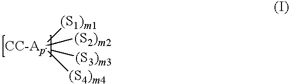

where CC is a predominantly planar carbon-conjugated core; A is an hetero-atomic group; p is 0, 1, 2, 3, 4, 5, 6, 7, or 8; S₁, S₂, S₃, and S₄ are substituents; m1, m2, m3 and m4 are 0, 1, 2, 3, 4, 5, 6, 7, or 8; and; sum (m1+m2+m3+m4) is 1, 2, 3, 4, 5, 6, 7, 8, 9 or 10.

In another embodiment of the disclosed film, the organic compound comprises rylene fragments. Examples of such organic compound include structures 1-23 shown in the Table 1.

TABLE 1
Examples of organic compound with rylene fragments

| | |
|---|---|
| 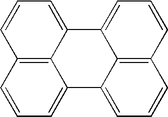 | 1 |
| 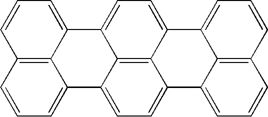 | 2 |
| 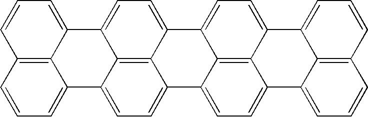 | 3 |
| 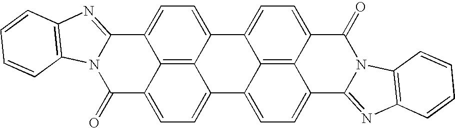 | 4 |
| 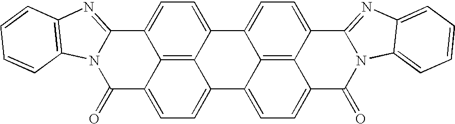 | 5 |
| 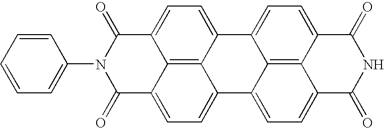 | 6 |
| 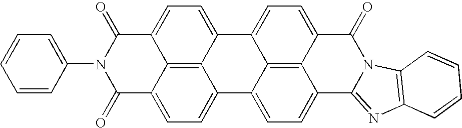 | 7 |
| 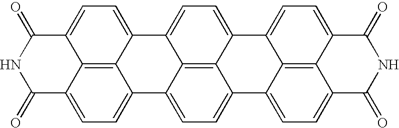 | 8 |
| 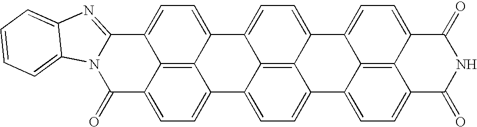 | 9 |
| 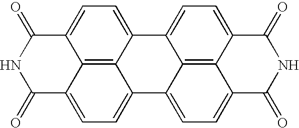 | 10 |
| 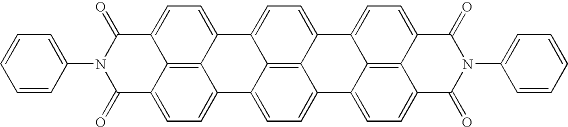 | 11 |
| 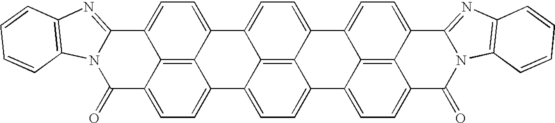 | 12 |
| 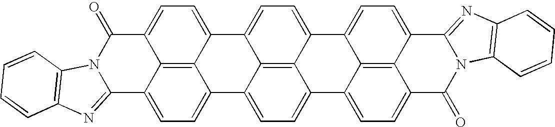 | 13 |
| 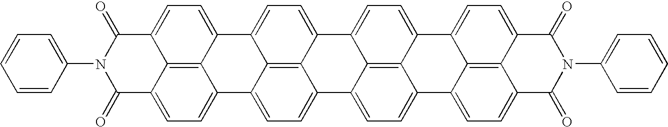 | 14 |
| 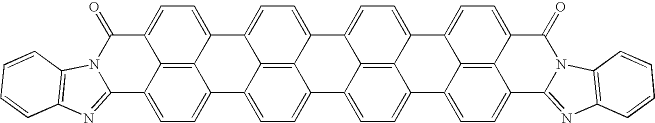 | 15 |
| 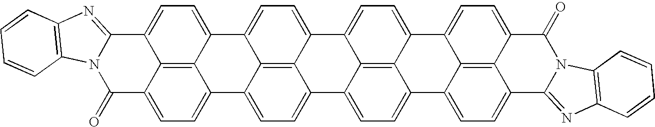 | 16 |
| 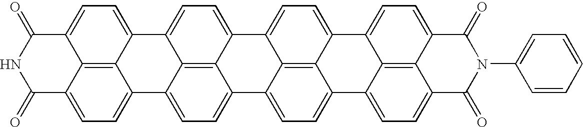 | 17 |
| 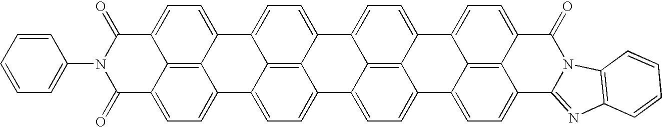 | 18 |
| 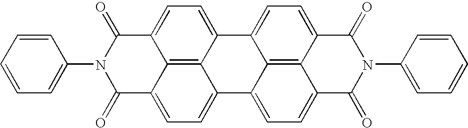 | 19 |
| 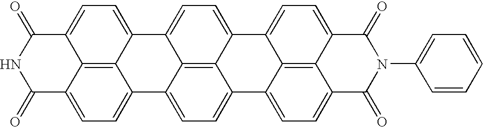 | 20 |
| 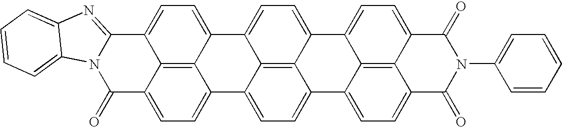 | 21 |
| 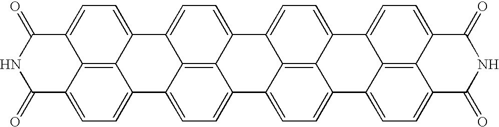 | 22 |
| 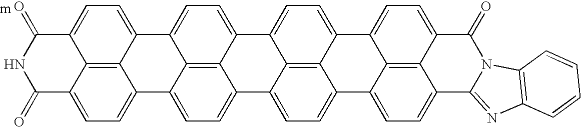 | 23 |

In still another embodiment of the disclosed film, the organic compound comprises one or more anthrone fragments. Examples of such organic compound include structures 24-31 shown in Table 2.

TABLE 2
Examples of organic compound with anthrone fragments

| | |
|---|---|
| 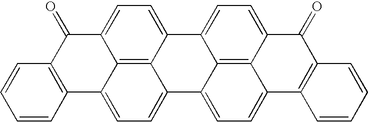 | 24 |
| 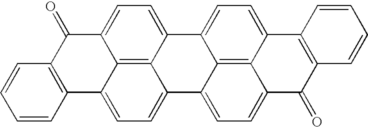 | 25 |
| 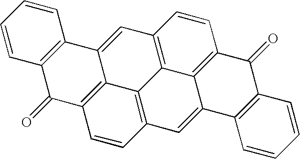 | 26 |
| 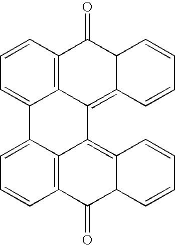 | 27 |
| 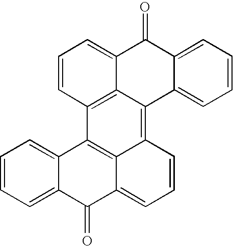 | 28 |
| 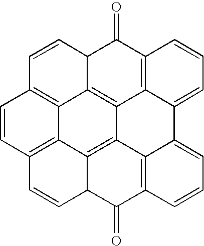 | 29 |
| 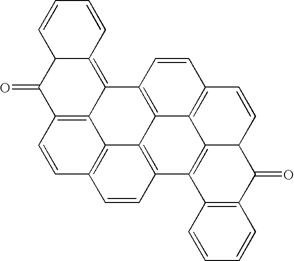 | 30 |
| 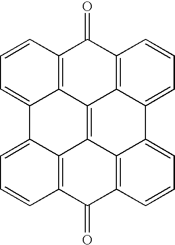 | 31 |

In yet another embodiment of the disclosed film, the organic compound comprises fused polycyclic hydrocarbons. Examples of such organic compound include structures 32-43 shown in Table 3. The fused polycyclic hydrocarbons are selected from the list comprising truxene, decacyclene, antanthrene, hexabenzotriphenylene, 1.2,3.4,5.6,7,8-tetra-(peri-naphthylene)-anthracene, dibenzoctacene, tetrabenzoheptacene, peropyrene, hexabenzocoronene, violanthrene, isoviolanthrene.

TABLE 3
Examples of organic compound with fused polycyclic hydrocarbons

| | |
|---|---|
| 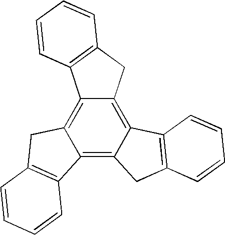 | 32 |
| 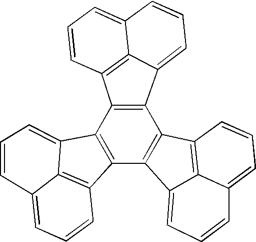 | 33 |
| 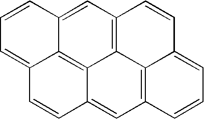 | 34 |
| 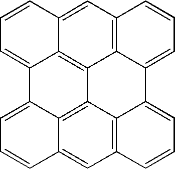 | 35 |
| 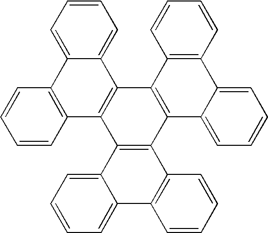 | 36 |
| 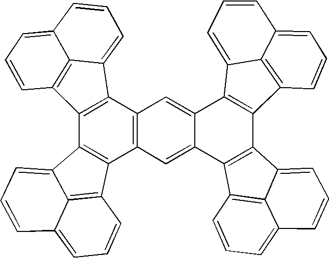 | 37 |
| 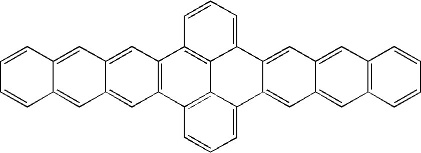 | 38 |
| 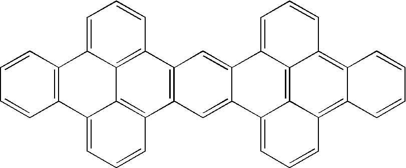 | 39 |
| 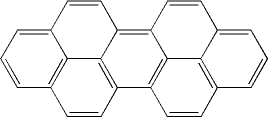 | 40 |
| 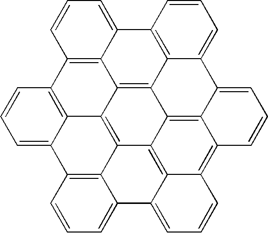 | 41 |
| 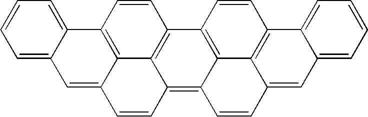 | 42 |
| 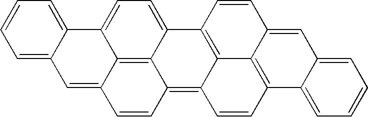 | 43 |

In one embodiment of the disclosed film, the organic compound comprises one or more coronene fragments. Examples of such organic compound include structures 44-51 shown in Table 4.

TABLE 4
Examples of organic compound with coronene fragments

| | |
|---|---|
| 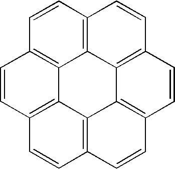 | 44 |
| 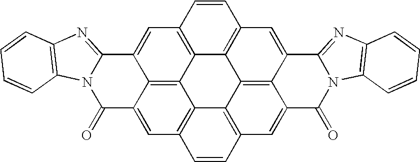 | 45 |
| 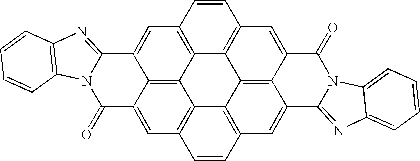 | 46 |
| 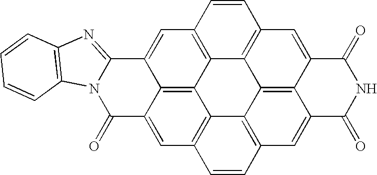 | 47 |
| 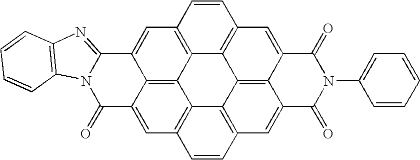 | 48 |
| 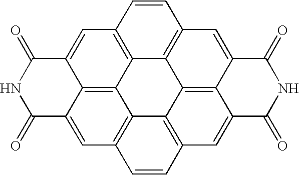 | 49 |
| 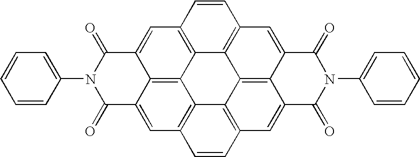 | 50 |
| 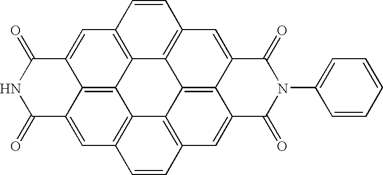 | 51 |

In one embodiment of the disclosed film, at least one of the hetero-atomic groups A is selected from the list comprising imidazole group, benzimidazole group, amide group, substituted amide group, and hetero-atom selected from nitrogen, oxygen, and sulfur.

In another embodiment of the disclosed film, at least one of the substituents S₁, S₂, S₃ and S₄ provides solubility of the organic compound in water or aqueous solution and is selected from the list comprising COO⁻, SO₃⁻, HPO₃⁻, and PO₃²⁻ and any combination thereof. In still another embodiment of the disclosed film, at least one of the substituents S₁, S₂, S₃ and S₄ provides solubility of the organic compound in the organic solvent and is selected from the list comprising CONR¹R², CONHCONH₂, SO₂NR¹R², R³, or any combination thereof, wherein R¹, R² and R³ are selected from hydrogen, an alkyl group, an aryl group, and any combination thereof, where the alkyl group has the general formula CₙH₂ₙ₊₁—where n is 1, 2, 3 or 4, and the aryl group is selected from the list comprising phenyl, benzyl and naphthyl. In yet another embodiment of the disclosed film, at least one of the substituents S₁, S₂, S₃ and S₄ provides solubility of the organic compound in organic solvents and is selected from the list comprising (C₁-C₃₅)alkyl, (C₂-C₃₅)alkenyl, and (C₂-C₃₅)alkinyl. In one embodiment of the disclosed film, at least one of the substituents S₁, S₂, S₃ and S₄ provides solubility of the organic compound in organic solvents and comprises fragments selected from the list comprising structures 52-58 shown in Table 5, where R is selected from the list, comprising linear or branched (C₁-C₃₅) alkyl, (C₂-C₃₅)alkenyl, and (C₂-C₃₅)alkinyl

TABLE 5
Examples of fragments of the substituents providing solubility

| | |
|---|---|
| 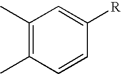 | 52 |
| 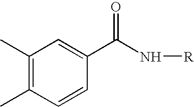 | 53 |
| 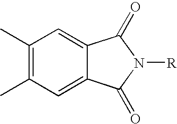 | 54 |
| 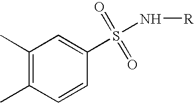 | 55 |
| 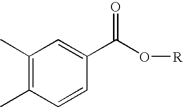 | 56 |
| 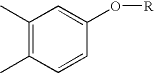 | 57 |
| 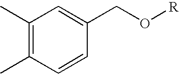 | 58 |

In another embodiment of the disclosed film, the organic solvent is selected from the list comprising ketones, carboxylic acids, hydrocarbons, chlorohydrocarbons, alcohols, ethers, esters, and any combination thereof. In still another embodiment of the disclosed film, the organic solvent is selected from the list comprising acetone, xylene, toluene, ethanol, methylcyclohexane, ethyl acetate, diethyl ether, octane, chloroform, methylenechloride, dichloroethane, trichloroethene, tetrachloroethene, carbon tetrachloride, 1,4-dioxane, tetrahydrofuran, pyridine, triethylamine, nitromethane, acetonitrile, dimethylformamide, dimethulsulfoxide, and any combination thereof.

In yet another embodiment of the disclosed film, at least one of the substituents S₁, S₂, S₃ and S₄ is a molecular binding group which number and arrangement provide for the formation of planar supramolecules from the organic compound molecules in the solution via non-covalent chemical bonds. In one embodiment of the disclosed film, at least one said binding group is selected from the list comprising a hydrogen acceptor (A_{H}), a hydrogen donor (D_{H}), and a group having the general structural formula

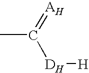

wherein the hydrogen acceptor (A_{H}) and hydrogen donor (D_{H}) are independently selected from the list comprising NH-group, and oxygen (O). In another embodiment of the disclosed film, at least one of the binding groups is selected from the list comprising hetero-atoms, COOH, SO₃H, H₂PO₃, NH, NH₂, CO, OH, NHR, NR, COOMe, CONH₂, CONHNH₂, SO₂NH₂, —SO₂—NH—SO₂—NH₂ and any combination thereof, where radical R is an alkyl group or an aryl group, the alkyl group having the general formula CₙH₂ₙ₊₁—where n is 1, 2, 3 or 4, and the aryl group being selected from the list comprising phenyl, benzyl and naphthyl.

In still another embodiment of the disclosed film, at least one of the substituents S₁, S₂, S₃ and S₄ is selected from the list comprising —NO₂, —Cl, —Br, —F, —CF₃, —CN, —OCH₃, —OC₂H₅, —OCOCH₃, —OCN, —SCN, and —NHCOCH₃.

In another preferable embodiment of the present invention, the device comprises at least one optically transparent and electrically conductive layer based on a ribtan material. In one embodiment of the disclosed device, at least one of the optically transparent and electrically conductive layers is transparent in the UV, visible and near IR regions of optical spectrum. In another embodiment of the disclosed device, the optically transparent and electrically conductive possesses polarizing properties in the visible spectral range. In still another embodiment of the disclosed device, the optically transparent and electrically conductive layer serves as electrode. In one embodiment of the present invention, the device is selected from the list comprising an optoelectronic device, a touch screen, an electromagnetic shield, a sensor, and a liquid-crystal display. In yet another embodiment of the disclosed device, at least one optically transparent and electrically conductive layer has an optical transparency of at least 80% for 550 nm light and a resistivity of less than 0.002-0.029 Ohm·cm. In one embodiment of the disclosed device, the ribtan material is prepared using at least one π-conjugated organic compound of the general structural formula I or a combination of the organic compounds of the general structural formula I:

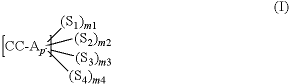

where CC is a predominantly planar carbon-conjugated core; A is an hetero-atomic group; p is 0, 1, 2, 3, 4, 5, 6, 7, or 8; S₁, S₂, S₃, and S₄ are substituents; m1, m2, m3 and m4 are 0, 1, 2, 3, 4, 5, 6, 7, or 8; and sum (m1+m2+m3+m4) is 1, 2, 3, 4, 5, 6, 7, 8, 9 or 10.

In one embodiment of the disclosed device, the organic compound comprises one or more rylene fragments. Examples of these organic compounds I-23 are given in Table 1.

In another embodiment of the disclosed device, the organic compound comprises one or more anthrone fragments. Examples of those organic compounds 24-31 are given in Table 2.

In still another embodiment of the disclosed device, the organic compound comprises planar fused polycyclic hydrocarbons. Examples of these hydrocarbons include truxene, decacyclene, antanthrene, hexabenzotriphenylene, 1.2,3.4,5.6,7,8-tetra-(peri-naphthylene)-anthracene, dibenzoctacene, tetrabenzoheptacene, peropyrene, hexabenzocoronene, violanthrene, isoviolanthrene (structures 32-43), as given in Table 3.

In yet another embodiment of the disclosed device, the organic compound comprises one or more coronene fragments. Examples of these organic compounds 44-51 are given in Table 4.

In one embodiment of the disclosed device, at least one of the hetero-atomic groups A is selected from the list comprising imidazole group, benzimidazole group, amide group, substituted amide group, and hetero-atom selected from nitrogen, oxygen, and sulfur.

In another embodiment of the disclosed device, at least one of the substituents S₁, S₂, S₃ and S₄ provides solubility of the organic compound in water or aqueous solution and is selected from the list comprising COO⁻, SO₃⁻, HPO₃⁻, and PO₃²⁻ and any combination thereof. In still another embodiment of the disclosed device, at least one of the substituents S₁, S₂, S₃ and S₄ provides solubility of the organic compound in the organic solvent and is selected from the list comprising CONR¹R², CONHCONH₂, SO₂NR¹R², R³, or any combination thereof, wherein R¹, R² and R³ are selected from hydrogen, an alkyl group, an aryl group, and any combination thereof, where the alkyl group has the general formula C₁H₂ₙ₊₁—where n is 1, 2, 3 or 4, and the aryl group is selected from the list comprising phenyl, benzyl and naphthyl. In yet another embodiment of the disclosed device, at least one of the substituents S₁, S₂, S₃ and S₄ provides solubility of the organic compound in organic solvents and is selected from the list comprising (C₁-C₃₅)alkyl, (C₂-C₃₅)alkenyl, and (C₂-C₃₅)alkinyl.

In one embodiment of the disclosed device, at least one of the substituents S₁, S₂, S₃ and S₄ provides solubility of the organic compound in organic solvents and comprises fragments selected from the list comprising structures 52-58 shown in Table 5, where R is selected from the list, comprising linear or branched (C₁-C₃₅) alkyl, (C₂-C₃₅)alkenyl, and (C₂-C₃₅)alkinyl.

In another embodiment of the disclosed device, the organic solvent is selected from the list comprising ketones, carboxylic acids, hydrocarbons, chlorohydrocarbons, alcohols, ethers, esters, and any combination thereof. In still another embodiment of the disclosed device, the organic solvent is selected from the list comprising acetone, xylene, toluene, ethanol, methylcyclohexane, ethyl acetate, diethyl ether, octane, chloroform, methylenechloride, dichloroethane, trichloroethene, tetrachloroethene, carbon tetrachloride, 1,4-dioxane, tetrahydrofuran, pyridine, triethylamine, nitromethane, acetonitrile, dimethylformamide, dimethulsulfoxide, and any combination thereof.

In yet another embodiment of the disclosed device, at least one of the substituents S₁, S₂, S₃ and S₄ is a molecular binding group which number and arrangement provide for the formation of planar supramolecules from the organic compound molecules in the solution via non-covalent chemical bonds. In one embodiment of the disclosed device, at least one binding group is selected from the list comprising a hydrogen acceptor (A_{H}), a hydrogen donor (D_{H}), and a group having the general structural formula:

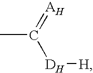

wherein the hydrogen acceptor (A_{H}) and hydrogen donor (D_{H}) are independently selected from the list comprising NH-group, and oxygen (O). In another embodiment of the disclosed device, at least one of the binding groups is selected from the list comprising hetero-atoms, COOH, SO₃H, H₂PO₃, NH, NH₂, CO, OH, NHR, NR, COOMe, CONH₂, CONHNH₂, SO₂NH₂, —SO₂—NH—SO₂—NH₂ and any combination thereof, where radical R is an alkyl group or an aryl group, the alkyl group having the general formula CₙH₂ₙ₊₁—where n is 1, 2, 3 or 4, and the aryl group being selected from the list comprising phenyl, benzyl and naphthyl.

In still another embodiment of the disclosed device, at least one of the substituents S₁, S₂, S₃ and S₄ is selected from the list comprising —NO₂, —Cl, —Br, —F, —CF₃, —CN, —OCH₃, —OC₂H₅, —OCOCH₃, —OCN, —SCN, and —NHCOCH₃.

The present invention also provides a method of producing at least one ribtan layer on a substrate, as disclosed hereinabove. Disclosed method comprises the following steps: (a) application of a solution of at least one π-conjugated organic compound of the general structural formula I or a combination of the organic compounds of the general structural formula I on a substrate:

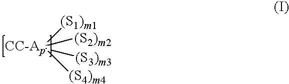

where CC is a predominantly planar carbon-conjugated core; A is a hetero-atomic group; p is 0, 1, 2, 3, 4, 5, 6, 7, or 8; S₁, S₂, S₃, and S₄ are substituents, m1, m2, m3 and m4 are 0, 1, 2, 3, 4, 5, 6, 7, or 8; and sum (m1+m2+m3+m4) is 1, 2, 3, 4, 5, 6, 7, 8, 9 or 10; (b) drying with formation of a solid precursor layer, and (c) formation of a ribtan layer. Said formation step is characterized by a level of vacuum, a composition and pressure of ambient gas, and a time dependence of temperature which are selected so as to ensure a creation of predominantly planar graphene-like structures in the ribtan layer. At least one said graphene-like structure possesses conductivity and is predominantly continuous within the entire ribtan layer. The thickness of the ribtan layer is in the range from approximately 1 nm to 1000 nm.

In one embodiment of the disclosed method, the predominantly planar carbon-conjugated core (CC), the substituents S₁, S₂, S₃, and S₄, and coating conditions are selected so that the graphene-like structures have form of planar graphene-like nanoribbons, the planes of which are oriented predominantly perpendicularly to the substrate surface. In another embodiment of the disclosed method, the predominantly planar carbon-conjugated core (CC), the substituents S₁, S₂, S₃, and S₄, and coating conditions are selected so that the graphene-like structures have form of planar graphene-like sheets the planes of which are oriented predominantly parallel to the substrate surface. In yet another embodiment of the disclosed method, the drying and formation steps are carried out simultaneously or sequentially. In still another embodiment of the disclosed method, the ambient gas comprises chemical elements selected from the list comprising hydrogen, nitrogen, fluorine, arsenic, boron, carbon tetrachloride, halogens, halogenated hydrocarbons, and any combination thereof. In one embodiment of the present invention, the disclosed method further comprises a post-treatment in a gas atmosphere. The post-treatment step is carried out after the formation step and the gas atmosphere comprises chemical elements selected from the list comprising hydrogen, nitrogen, fluorine, arsenic, boron, carbon tetrachloride, halogens, halogenated hydrocarbons, and any combination thereof. In another embodiment of the present invention, the disclosed method further comprises a doping step carried out after the formation step and/or after the post-treatment step and during which the ribtan layer is doped with impurities. The doping step is based on a method selected from the list comprising diffusion method, intercalation method or ion implantation method and the impurity is selected from the list comprising Sb, P, As, Ti, Pt, Au, O, B, Al, Ga, In, Pd, S, F, N, Br, I and any combination thereof. In one embodiment of the disclosed method, at least one of the hetero-atomic groups is selected from the list comprising imidazole group, benzimidazole group, amide group and substituted amide group. In another embodiment of the disclosed method, said solution is based on water and at least one of the substituents providing solubility of the organic compound is selected from the list comprising COO⁻, SO₃⁻, HPO₃⁻, and PO₃²⁻, and any combination thereof. In yet another embodiment of the disclosed method, said solution is based on organic solvent and wherein the organic solvent is selected from the list comprising ketones, carboxylic acids, hydrocarbons, cyclohydrocarbons, chlorohydrocarbons, alcohols, ethers, esters, acetone, xylene, toluene, ethanol, methylcyclohexane, ethyl acetate, diethyl ether, octane, chloroform, methylenechloride, dichloroethane, trichloroethene, tetrachloroethene, carbon tetrachloride, 1,4-dioxane, tetrahydrofuran, pyridine, triethylamine, nitromethane, acetonitrile, dimethylformamide, dimethulsulfoxide, and any combination thereof. At least one of the substituents providing solubility of the organic compound in the organic solvent is selected from the list comprising linear and branched (C₁-C₃₅)alkyl, (C₂-C₃₅)alkenyl, and (C₂-C₃₅)alkinyl, an amide of an acid residue independently selected from the list comprising CONR₁R₂, CONHCONH₂, SO₂NR₁R₂, R₃, and any combination thereof. The radicals R₁, R₂ and R₃ are independently selected from the list comprising hydrogen, a linear alkyl group, a branched alkyl group, an aryl group, and any combination thereof. The alkyl group comprises a general formula —(CH₂)ₙCH₃, where n is an integer from 0 to 27, and the aryl group is selected from the group comprising phenyl, benzyl and naphthyl. In yet another embodiment of the disclosed method, the organic compound further comprises at least one bridging group B_{G} to provide a connection between at least one of the substituents providing solubility of the organic compound in the organic solvent and the predominantly planar carbon-conjugated core and wherein at least one of the bridging groups B_{G} is selected from the list, comprising C(O)—, C(O)O—, —C(O)—NH—, —(SO₂)NH—, —O—, —CH2O—, —NH—, >N—, and any combination thereof.

In one embodiment of the disclosed method, said organic compound comprises rylene fragments having a general structural formula from the group comprising structures 1-23 shown in Table 1. In another embodiment of the disclosed method, said organic compound comprises anthrone fragments having a general structural formula from the group comprising structures 24-31 shown in Table 2. In yet another embodiment of the disclosed method, said organic compound comprises fused polycyclic hydrocarbons selected from the list comprising truxene, decacyclene, antanthrene, hexabenzotriphenylene, 1.2,3.4,5.6,7,8-tetra-(peri-naphthylene)-anthracene, dibenzoctacene, tetrabenzoheptacene, peropyrene, hexabenzocoronene, violanthrene, isoviolanthrene and having a general structural formula from the group comprising structures 32-43 shown in Table 3. In still another embodiment of the disclosed method, said organic compound comprises coronene fragments having a general structural formula from the group comprising structures 44-51 shown in Table 4. In one embodiment of the disclosed method, said drying stage is carried out using airflow. In another embodiment of the present invention, the disclosed method further comprises a pre-treatment of the substrate prior to the application of said solution so as to render its surface hydrophilic. In yet another embodiment of the disclosed method, a type of the solution is selected from the list comprising an isotropic solution and a lyotropic liquid crystal solution. In still another embodiment of present invention, the disclosed method further comprises an alignment action, wherein the alignment action is simultaneous or subsequent to the application of said solution on the substrate. In one embodiment of the disclosed method, said application stage is carried out using a technique selected from the list comprising a spray-coating, Mayer rod technique, blade coating, slot-die application, extrusion, roll coating, curtain coating, knife coating, and printing. In another embodiment of the disclosed method, the π-conjugated organic compound further comprise molecular binding groups which number and arrangement thereof provide for the formation of planar supramolecules from the organic compound molecules in the solution via non-covalent chemical bonds. At least one said binding groups is selected from the list comprising hetero-atoms, COOH, SO₃H, H₂PO₃, NH, NH₂, CO, OH, NHR, NR, COOMe, CONH₂, CONHNH₂, SO₂NH₂, —SO₂—NH—SO₂—NH₂, and any combination thereof, a hydrogen acceptor (A_{H}), a hydrogen donor (D_{H}), and a group having a general structural formula

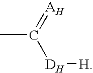

The radical R is independently selected from the list comprising a linear alkyl group, a branched alkyl group, and an aryl group, and any combination thereof, where the alkyl group has the general formula —(CH₂)ₙCH₃, where n is an integer from 0 to 27, and where the aryl group is selected from the group comprising phenyl, benzyl and naphthyl. The hydrogen acceptor (A_{H}) and hydrogen donor (D_{H}) are independently selected from the list comprising NH-group, and oxygen (O). The non-covalent chemical bonds are independently selected from the list comprising a single hydrogen bond, dipole-dipole interaction, cation—pi-interaction, Van-der-Waals interaction, coordination bond, ionic bond, ion-dipole interaction, multiple hydrogen bond, interaction via the hetero-atoms, and any combination thereof and the planar supramolecule have the form selected from the list comprising disk, plate, lamella, ribbon, and any combination thereof. In another embodiment of the disclosed method, the rod-like supramolecules are predominantly oriented in the plane of the substrate.

In yet another embodiment of the disclosed method, the formation step is carried out in vacuum or an inert gas. In still another embodiment of the disclosed method, the formation step is carried out as process of annealing so as to ensure 1) partial pyrolysis of the organic compound with at least partial removing of substituents, hetero-atomic and solubility groups from the solid precursor layer, and 2) fusion of the carbon-conjugated residues. In one embodiment of the disclosed method, the pyrolysis temperature is in the range between approximately 150 and 650 degrees C. and the fusion temperature is in the range between approximately 500 and 2500 degrees C. In another embodiment of the disclosed method, the formation step is carried out without heating or under moderate heating (less than 500 degrees C) under the action of gas-phase or liquid phase environment containing molecules which are sources of free radicals or benzyne fragments. In yet another embodiment of the disclosed method, said formation step is further accompanied by applying an external action upon the ribtan layer stimulating low-temperature carbonization process and formation of the graphene-like carbon-based structures.

In one embodiment of the present invention, the disclosed method further comprises the step of removing the substrate by one of the methods selected from the list comprising wet chemical etching, dry chemical etching, plasma etching, laser etching, grinding, and any combination thereof.

In yet another embodiment of the disclosed method, the substituents S₁, S₂, S₃, and S₄ comprises identical substituents providing solubility of the organic compound or the substituents S₁, S₂, S₃, and S₄ comprises more than two substituents providing solubility of the organic compound and at least one substituent is different from the other or others. In still another embodiment of the disclosed method, the steps (a), (b) and (c) are consistently repeated two or more times, and sequential ribtan layers are formed using solutions based on the same or different organic compounds or their combinations. In one embodiment of the disclosed method, at least one said π-conjugated organic compound further comprises substituents independently selected from a list comprising —NO₂, —Cl, —Br, —F, —CF₃, —CN, —OH, —OCH₃, —OC₂H₅, —OCOCH₃, —OCN, —SCN, —NH₂, —NHCOCH₃, and —CONH₂.

The present invention also provides a method for producing a ribtan layer on a substrate, as disclosed hereinabove. The disclosed method comprises the following steps: (a) preparation of a solution of one π-conjugated organic compound of a general structural formula II or a combination of the organic compounds of the general structural formula II capable of forming supramolecules:

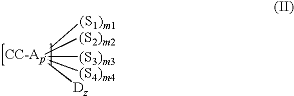

where CC is a predominantly planar carbon-conjugated core; A is an hetero-atomic group; p is 0, 1, 2, 3, 4, 5, 6, 7, or 8; S₁, S₂, S₃, S₄ and D are substituents, where S₁, S₂, S₃, and S₄ are substituents providing a solubility of the organic compound in suitable solvent and D is a substituent which produces reaction centers selected from the list comprising free radicals and benzyne fragments on the predominantly planar carbon-conjugated cores after a subsequent elimination of this substituent during a step (e); m1, m2, m3 and m4 are 0, 1, 2, 3, 4, 5, 6, 7, or 8; sum (m1+m2+m3+m4) is 1, 2, 3, 4, 5, 6, 7, 8, 9 or 10, and z is 0, 1, 2, 3 or 4; (b) deposition of a layer of the solution on the substrate; (c) an alignment action upon the solution in order to ensure preferred alignment of the supramolecules; (d) drying with formation of a solid precursor layer; and (e) application of an external action upon the solid precursor layer stimulating low-temperature carbonization and formation of the ribtan layer. In one embodiment of the disclosed method, the substituent D is selected from the list comprising halogens Cl, Br and I. In another embodiment of the disclosed method, said deposition step is carried out using a technique selected from the list comprising a spray-coating, Mayer rod technique, blade coating, slot-die application, extrusion, roll coating, curtain coating, knife coating, and printing. In yet another embodiment of the disclosed method, the alignment action upon the surface of the solution layer is produced by a directed mechanical motion of at least one aligning instrument selected from the list comprising a knife, cylindrical wiper, flat plate and any other instrument oriented parallel to the deposited solution layer surface, whereby a distance from the substrate surface to the edge of the aligning instrument is preset so as to obtain a solid precursor layer of a required thickness. In still another embodiment of the disclosed method, the alignment action is performed by using techniques selected from the list comprising a heated instrument, application of an external electric field to the deposited solution layer, application of an external magnetic field to the deposited solution layer, application of an external electric and magnetic field to the deposited solution layer, with simultaneous heating, illuminating the deposited solution layer with at least one coherent laser beams, and any combination of the above listed techniques. In one embodiment of the disclosed method, the external action is selected from the list comprising a thermal treatment and an ultraviolet irradiation. In another embodiment of the disclosed method, the thermal treatment is carried out at the temperature not exceeding the melting temperature of a substrate material.

In yet another embodiment of the disclosed method, said organic compound comprises rylene fragments having a general structural formula from the group comprising structures 1-23 shown in Table 1. In still another embodiment of the disclosed method, said organic compound comprises anthrone fragments having a general structural formula from the group comprising structures 24-31 shown in Table 2. In one embodiment of the disclosed method, said organic compound comprises fused polycyclic hydrocarbons selected from the list comprising truxene, decacyclene, antanthrene, hexabenzotriphenylene, 1.2,3.4,5.6,7,8-tetra-(peri-naphthylene)-anthracene, dibenzoctacene, tetrabenzoheptacene, peropyrene, hexabenzocoronene, violanthrene and isoviolanthrene and having a general structural formula from the group comprising structures 32-43 shown in Table 3. In another embodiment of the disclosed method, said organic compound comprises coronene fragments having a general structural formula from the group comprising structures 44-51 shown in Table 4. In yet another embodiment of the present invention, the disclosed method further comprises a step of introduction (placement, location) the solid layer into gas-phase environment containing molecules which are sources of free radicals or benzyne fragments, wherein this additional step follows after the drying step.

The present invention will now be described more fully hereinafter with reference to the following example, in which preferred embodiments of the present invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art. In the drawings, the thickness of layers and regions are exaggerated for clarity.

### EXAMPLES

### Example 1

The example describes synthesis of bis(carboxybenzimidazoles) of perylene tetracarboxylic acid (rylene fragments 4 and 5 in Table 1):

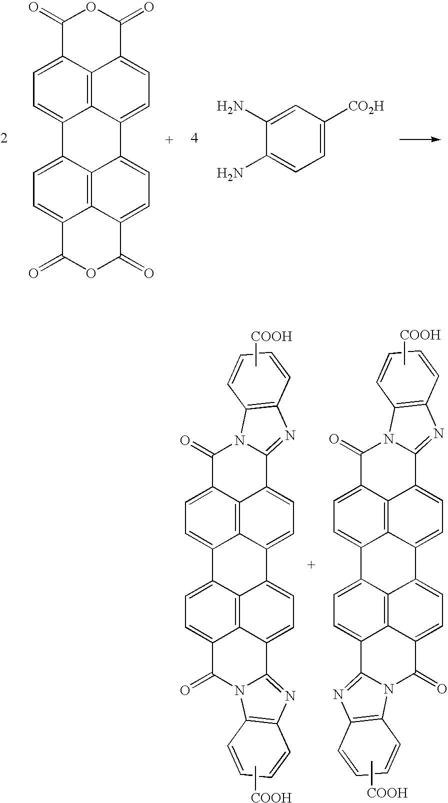

Mixture of 3,4,9,10-perylenetetracarboxylic-3,4:9,10-dianhydride (10 g) and 3,4-diaminobenzoic acid (39 g) was agitated in N-methylpyrrolidone (250 ml) for 6 hours at 175-180° C. A self cooled reaction mass was filtered. Filter cake was rinsed with N-methylpyrrolidone and dissolved in the mixture of water (1500 ml) and concentrated ammonia solution (100 ml). Dimethylformaide (1 l) was added to the solution. Precipitate was filtered and rinsed with dimethylformaide. Filter cake was suspended in water (1 l). Concentrated hydrochloric acid (100 ml) was added and the precipitate was filtered. The obtained filter cake was suspended in ˜500 ml of water, filtered and rinsed with water. Yield 13.2 g.

### Example 2

The example describes synthesis of diphenylimide of 3,4,9,10-perylenetetracarboxylic acid (rylene fragment 19 in Table 1):

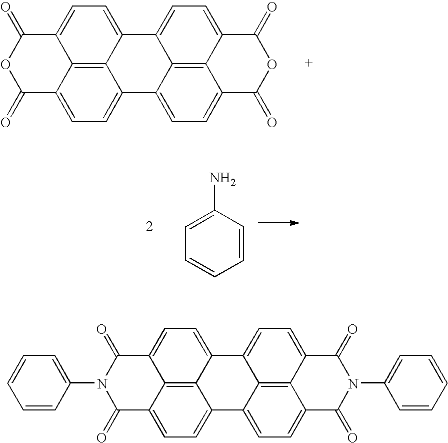

Mixture of 3,4,9,10-perylenetetracarboxylic-3,4:9,10-dianhydride (40 g), aniline (38 ml), zinc chloride (21 g) and ethylene glycol (400 ml) was agitated 8 hours at 180-185° C. After a self cooling process a precipitate was filtered and rinsed with hot water (1 l). Filter cake was agitated in a 1% solution of potassium hydroxide for 2 hours. Precipitate was filtered and rinsed with hot water (1 l). Filter cake was agitated in a 2% solution of hydrogen chloride for 1 hour at 90° C. Precipitate was filtered and rinsed with hot water (1 l). Filter cake was agitated in a 1% solution of potassium hydroxide for 2 hours. Precipitate was filtered and rinsed with hot water (1 l). Filter cake was agitated in a 2% solution of hydrogen chloride for 1 hour at 90° C. Precipitate was filtered and rinsed with hot water (1 l) and dried at 100° C. Yield 38.3 g.

### Example 3

The example describes synthesis of dicarboxymethylimide of perylentetracarboxylic acid (carboxylic acid of base rylene fragment 10 in the Table 1)

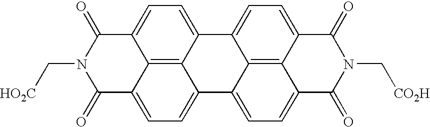

Mixture of 3,4,9,10-perylenetetracarboxylic-3,4:9,10-dianhydride (2 g) and glycine (3.8 g) was agitated in the boiling N-methylpyrrolidone (50 ml) for 6 hours. A self cooled reaction mass was filtered. Filter cake was rinsed with N-methylpyrrolidone, hydrochloric acid and water. Obtained filter cake was suspended in ˜300 ml of water, filtered and rinsed with water. Yield 0.73 g.

### Example 4

The example describes synthesis of violanthrone disulfonic acid (anthrone fragment 24 in Table 2):

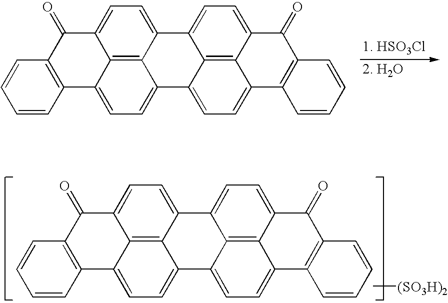

Violanthrone (10 g) was added to chlorosulfonic acid (50 ml) at ambient conditions. Then reaction mass was agitated at 85-90° C. for 15 hours. After self cooling a reaction mass was added by parts into water (600 ml). Precipitate was filtered and rinsed with water until filtrate became colored. Filter cake was agitated in the boiling water (500 ml) for two hours. The product was precipitated by addition of concentrated hydrochloric acid (600 ml). Precipitate was filtered, washed with 6 N hydrochloric acid (200 ml) and dried in oven (˜100° C.). Yield 11.8 g.

### Example 5

The example describes synthesis of isoiolanthrone disulfonic acid (anthrone fragment 25 in Table 2):

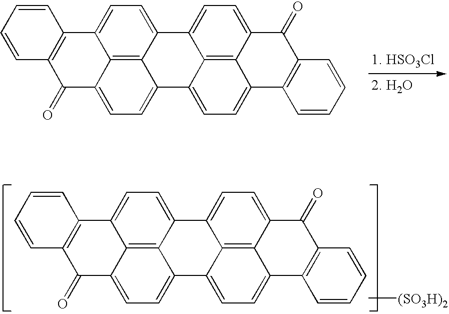

Isoviolanthrone (10 g) was charged into chlorosulfonic acid (50 ml) at ambient conditions. Then reaction mass was agitated at 85-90° C. for 16 hours. After self cooling a reaction mass was added into water (600 ml) by portions. Filter cake was agitated in the boiling water (600 ml) for 3 hours. The obtained hot solution was filtered through fiber glass filters. The substance was precipitated by addition of concentrated hydrochloric acid (550 ml). Precipitate was filtered, washed with 4 N hydrochloric acid (200 ml). Filter cake was suspended in 300 ml of 4 N hydrochloric acid. Precipitate was filtered, washed with 4 N hydrochloric acid (100 ml) and dried in oven (˜100° C.). Yield 7.5 g.

### Example 6

The example describes synthesis of decacyclene (polycyclic hydrocarbon fragment 33 in Table 3):

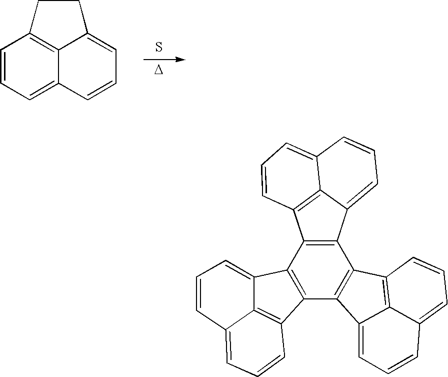

Mixture of sulfur powder (10 g), acenaphthene (31 g) and potassium hydroxide (0.4 g) was heated at 230-300° C. for 7 hours. Obtained fusion cake was ground and agitated in a boiling tetrachloroethane (200 ml) for 4 hours. Suspension was filtered at ˜80° C. Filter cake was agitated in a boiling tetrachloroethane (200 ml) for 2 hours. Cooled suspension was filtered. Filter cake was rinsed with tetrachloroethane and suspended in a hot N-methylpyrrolidone (300 ml, ˜150° C.). Cooled suspension was diluted with isopropanol (400 ml) and a precipitate was filtered. Filter cake was suspended in hot N-methylpyrrolidone (400 ml, ˜150° C.). Cooled suspension was filtered. Filtrate was diluted with water (1.5 l). Obtained precipitate was filtered, rinsed with water and dried at ˜100° C. 11.2 g of dry powder were prepared. Filtrate (N-methylpyrrolidone—isopropanol) was diluted with water (1 l). Precipitate was filtered, rinsed with water and dried at ˜100° C. 1.18 g of dry powder was prepared. Obtained powders were combined and agitated in the boiling tetrachloroethane (70 ml) for 2 hours. Cooled suspension was filtered. Filter cake was rinsed with tetrachloroethane and chloroform. Obtained powder (10.8 g) was suspended in hot N-methylpyrrolidone (400 ml, ˜150° C.). Cooled suspension was diluted with water (1 l). Obtained precipitate was filtered, rinsed with water and dried at ˜100° C. Yield 6.5 g.

### Example 7

The example describes synthesis of decacyclene trisulfonic acid (polycyclic hydrocarbon fragment 33 in Table 3):

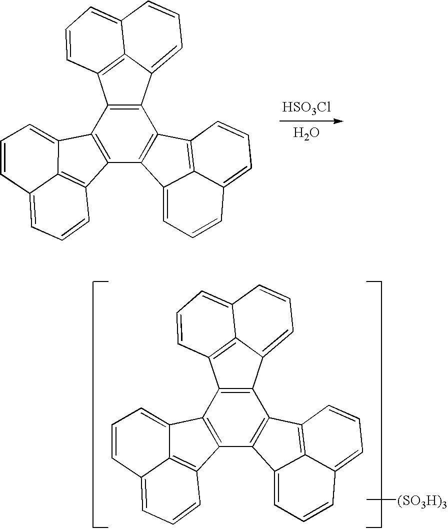

Decacyclene (1 g) was charged into chlorosulfonic acid (5 ml) at ambient conditions. During charging hydrogen chloride was liberating. Then reaction mass was agitated at the room temperature for 48 hours. After that a reaction mass was added into water (50 ml) by portions. Precipitate was filtered. Filter cake was agitated in water (100 ml) at ambient conditions and in hot water (80° C.) for 2 hours. Prepared solution was filtered through fiber glass filter. Filtrate was diluted with concentrated hydrochloric acid (100 ml) and dried at ˜100° C. Yield 1.13 g.

### Example 8

The example describes synthesis of truxene (polycyclic hydrocarbon fragment 32 in Table 3):

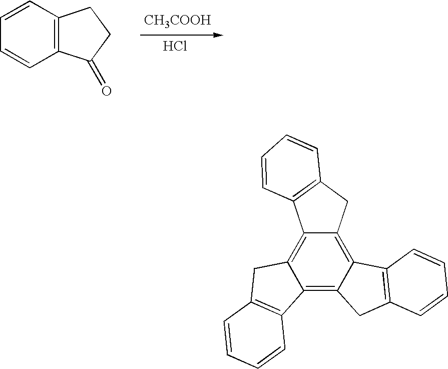

1-Indanone (5.0 g) was inserted into a mixture of acetic acid (22 mL) and concentrated hydrochloric acid (11 mL). The resultant solution was agitated at 95-97° C. for 16 hours. Color turned yellow, bulky precipitate formed. The precipitate was filtered off, the solid material was washed with water (2×100 mL) and with acetone (100 mL, cold 5-7° C.). Yield 3.2 g.

### Example 9

The example describes synthesis of truxene trisulfonic acid (polycyclic hydrocarbon fragment 32 in Table 3):

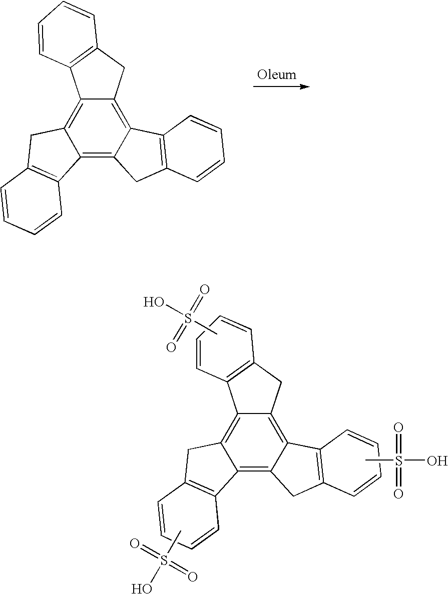

Truxene (3.4 g) was charged into oleum (80 mL, 4%), slowly for 15 min trying to keep particles of the substance as fine as possible. The outer water bath was used to insure the room temperature of reaction mixture. Reaction mass was agitated for 5 hours. After that it was added dropwise into ice (135 g). Pale-crème precipitate was diluted with concentrated hydrochloric acid (150 mL), stirred overnight, filtered off, then washed with concentrated hydrochloric acid (150 mL), water (60 mL) and the resultant solution was diluted with 36% hydrochloric acid (150 mL). A jelly brown-green jelly precipitate was formed, solution was removed, and a fresh portion of hydrochloric acid was added (150 mL). Stirring was continued whereas jelly mass turned to a solid precipitate. Then suspension was filtered, the solid material was washed with concentrated hydrochloric acid (50 mL), dried over wet sodium hydroxide, phosphorous oxide with mild heating. Yield 6.7 g.

### Example 10

This Example describes preparation of N,N′-(1-undecyl)dodecyl-5,11-dihexylcoronene-2,3:8,9-tetracarboxydiimide (coronene fragment 49 in the Table 4). The preparation comprised 6 steps:

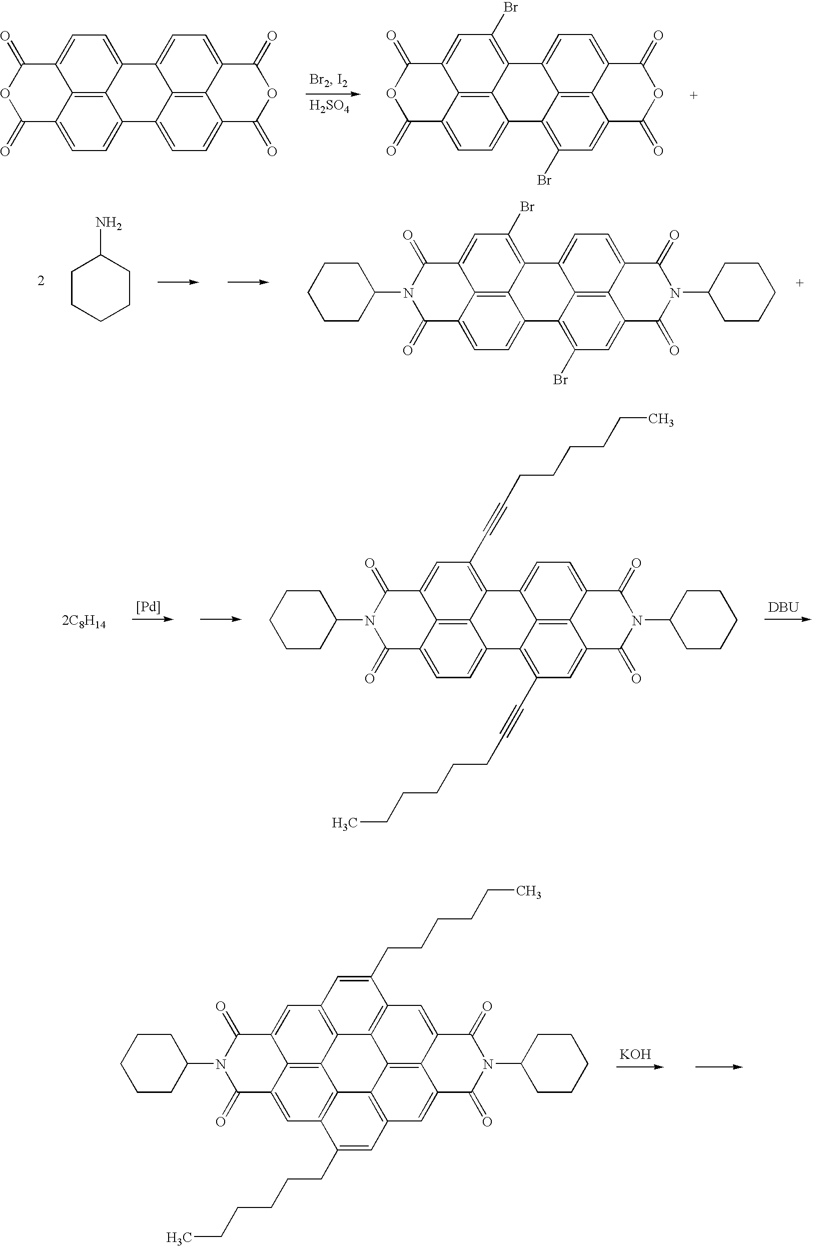

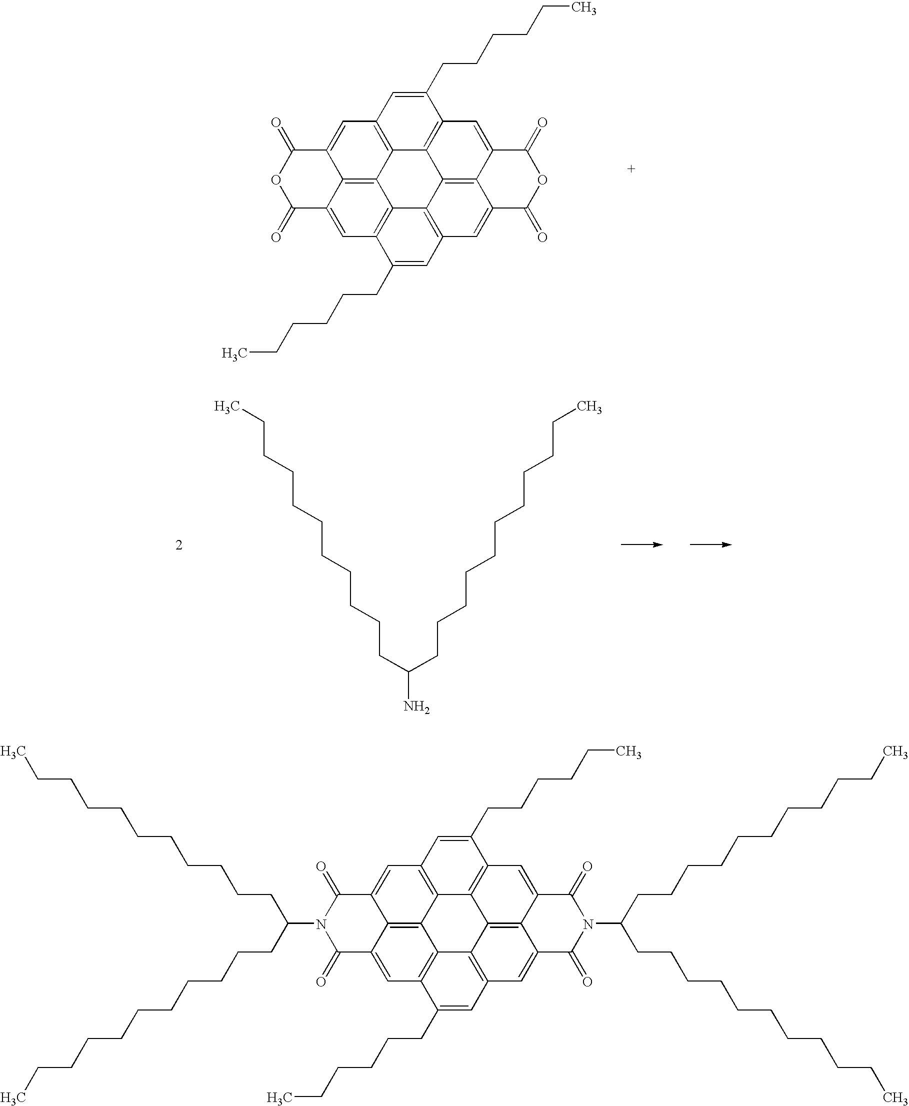

Commercially available perylene-3,4:9,10-tetracarboxylic dianhydride (100.0 g, 0.255 mol) was brominated with mixture of bromine (29 mL) and iodine (2.38 g) in 100% sulfuric acid (845 mL) at. ˜85° C. The yield of 1,7-Dibromoperylene-3,4:9,10-tetracarboxylic dianhydride was 90 g (64%).

Analysis: calculated: C₂₄H₆Br₂O₆, C, 52.40; H, 1.10; Br 29.05; O 17.45%. found: C, 52.29; H, 1.07; Br 28, 79%. Absorption spectrum (9.82×10⁻⁵ M solution in 93% sulfuric acid): 405 (9572), 516 (27892), 553 (37769).

N,N′-Dicyclohexyl-1,7-dibromoperylene-3,4:9,10-tetracarboxydiimide was synthesized by the reaction of 1,7-dibromoperylene-3,4:9,10-tetracarboxylic dianhydride (30.0 g) with cyclohexylamine (18.6 mL) in N-methylpyrrolidone (390 mL) at ˜85° C.

The yield of N,N′-dicyclohexyl-1,7-dibromoperylene-3,4:9,10-tetracarboxydiimide was 30 g (77%).

N,N′-dicyclohexyl-1,7-di(oct-1-ynyl)perylene-3,4:9,10-tetracarboxydiimide was synthesized by Sonagashira reaction: N,N′-dicyclohexyl-1,7-dibromoperylene-3,4:9,10-tetracarboxydiimide (24.7 g) and octyne-1 (15.2 g) in the presence of bis(triphenylphosphine)palladium(II) chloride (2.42 g), triphenylphospine (0.9 g), and copper(I) iodide (0.66 g). The yield of N,N′-dicyclohexyl-1,7-di(oct-1-ynyl)perylene-3,4:9,10-tetracarboxydiimide was 15.7 g (60%).

N,N′-dicyclohexyl-5,11-dihexylcoronene-2,3:8,9-tetracarboxydiimide was synthesized by the heating of N,N′-dicyclohexyl-1,7-di(oct-1-ynyl)perylene-3,4:9,10-tetracarboxydiimide (7.7 g) in toluene (400 mL) in the presence of 1,8-diazabicyclo[5.4.0]undec-7-ene (0.6 ml) at 100-110° C. for 20 hours.

5,11-dihexylcoronene-2,3:8,9-tetracarboxylic dianhydride was prepared by hydrolysis of N,N′-dicyclohexyl-5,11-dihexylcoronene-2,3:8,9-tetracarboxydiimide (6.4 g, 8.3 mmol) with potassium hydroxide (7.0 g, 85%) in the mixture of tert-butanol (400 mL) and water (0.4 mL) at 85-90° C. The yield of 5,11-dihexylcoronene-2,3:8,9-tetracarboxylic dianhydride was 4.2 g (83%).

N,N′-(1-undecyl)dodecyl-5,11-dihexylcoronene-2,3:8,9-tetracarboxydiimide was synthesized by the reaction of 5,11-di(hexyl)coronene-2,3:8,9-tetracarboxylic dianhydride with 12-tricosanamine.

5,11-di(hexyl)coronene-2,3:8,9-tetracarboxylic dianhydride (3.44 g), 12-tricosanamine (7.38 g), benzoic acid (45 mg) and 3-Chlorophenol (15 mL) were evacuated and saturated with argon two times at room temperature and then two times at 100° C. The reaction mixture was agitated at ˜140° C. for 1 hour and 160-165° C. for 20 hours in a flow of argon. After that the reaction mixture was agitated at ˜100° C. and was vacuumed at 10 mm Hg for half an hour. Then apparatus was filled with argon once again and heating was continued for the next 24 hours.

A drop of reaction mixture was mixed with acetic acid (5 mL), centrifuged, solid was dissolved in chloroform (0.5 mL) which was washed with water and dried over sodium sulfate. Thin layer chromatography probe showed good formation of product with Rf 0.9 (eluent: chloroform-hexane-ethylacetate-methanol (100:50:0.3:0.1 by V)).

The reaction mixture was added in small portions to acetic acid (500 mL) with simultaneous shaking. The orange-red suspension was kept for 3 hours with periodic shaking, then filtered off. The filter cake was washed with water (0.5 L), and then was shaken with water (0.5 L) and chloroform (250 mL) in a separator funnel. The organic layer was separated, washed with water (2×350 mL) and dried over sodium sulfate overnight. The evaporation resulted in 7.0 g of crude product.

Column chromatography was carried out using exactly tuned eluent mixture: chloroform (700 mL), petroleum ether (2 L), ethylacetate (0.6 mL), and methanol (0.2).

Column chromatography was carried out using column: l=20, d=7 cm. Elution of orange fraction and evaporation resulted in orange soft solid material, which was dissolved in chloroform (25 mL) and added slowly to methanol (400 mL) with agitation. The soft precipitate was dried on air overnight, then in vacuum (15 mm Hg) at mild heating) (35° for 5 hours. The yield of preparation of N,N′-(1-undecyl)dodecyl-5,11-dihexylcoronene-2,3:8,9-tetracarboxydiimide was 5.0 g (70%).

### Example 11

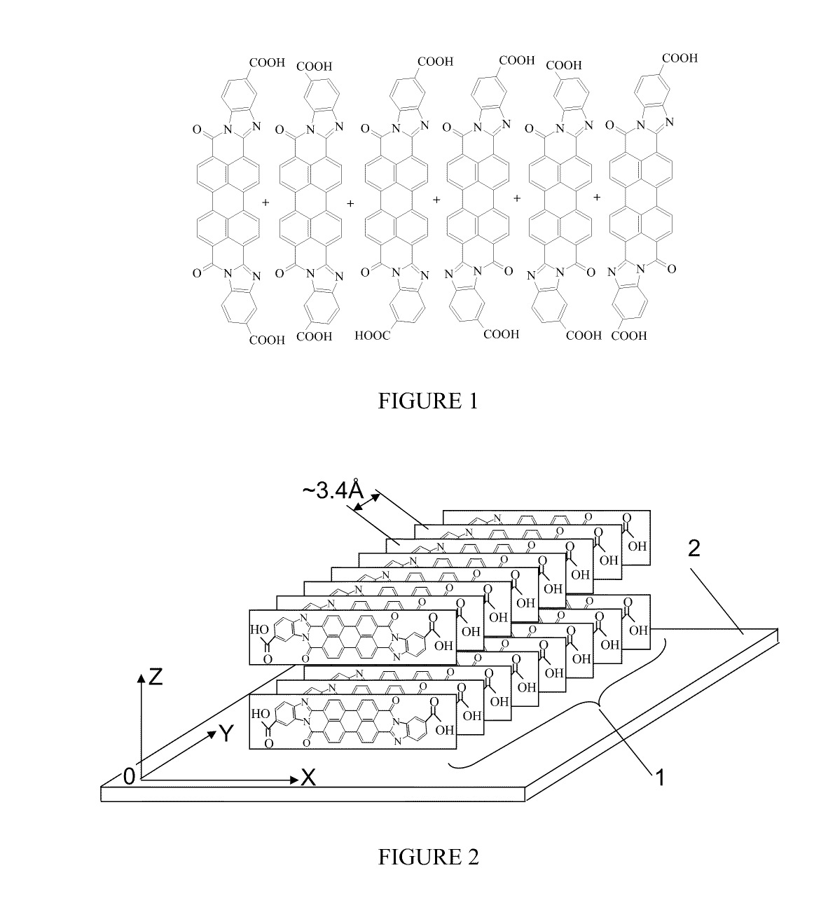

Example 11 describes a formation of the disclosed film. The ribtan layer comprising graphene-like carbon-based structures was formed by a mixture of bis(carboxybenzimidazoles) of prerylenetetracarboxylic acids (bis-carboxy DBI PTCA). As a first step, a water solution of bis-carboxy DBI PTCA was applied on a substrate. The solution comprised a mixture of six isomers as shown in FIG. 1, which predominantly planar carbon-conjugated cores are shown in Table 1, ##4 and 5. Bis-carboxy DBI PTCA is a π-conjugated organic compound, where the predominantly planar carbon-conjugated core (CC in formula I) comprises rylene fragments, the benzimidazole groups serve as hetero-atomic groups, and carboxylic groups serve as substituents providing solubility. The molecular structure provides for the formation of rod-like molecular stacks. In this Example quartz was used as a substrate material. The Mayer rod technique was used to coat the water-based solution of bis-carboxy DBI PTCA. During the second step the drying was performed at 40 degrees C. and humidity of approximately 70%. By the end of the drying step, the layer usually retained about 10% of the solvent. As a result of drying the layer comprised rod-like supramolecules oriented along the coating direction. FIG. 2 schematically shows the supramolecule (1) oriented along the y-axis and located on the substrate (2). Distance between the planes of bis-carboxy DBI PTCA is approximately equal to 3.4 A.

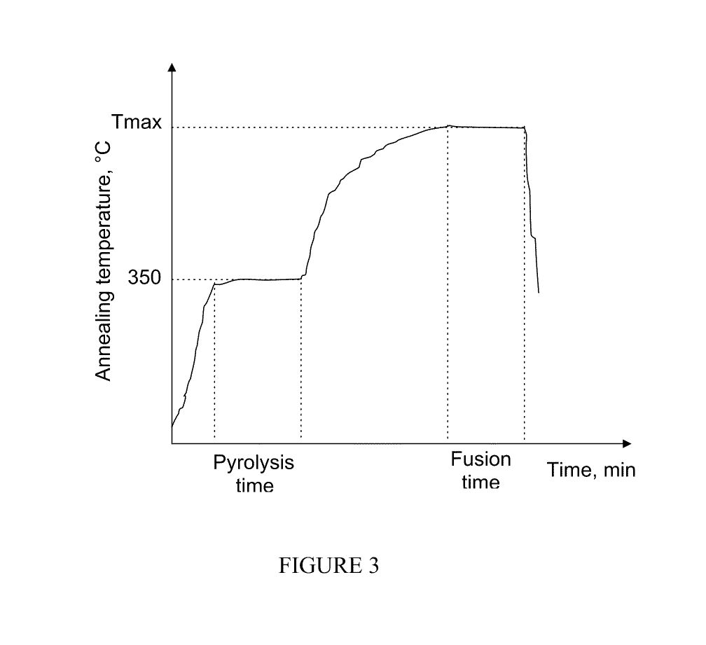

The annealing step was carried out in vacuum. The annealing step may be carried out in nitrogen or other inert gases flow. The annealing step included two steps, 1) exposure of bis-carboxy DBI PTCA film at 350° C. for 30 minutes in order to carry out partial pyrolysis of the organic compound with at least partial removal of the hetero-atomic groups and the substituents from the layer, and 2) fusion in vacuum of the carbon-conjugated residues at temperatures 720° C. for 60 minutes in order to generate the predominantly planar graphene-like carbon-based structures. The annealing regime is shown in FIG. 3. At least part of the substituents S₁, S₂, S₃ and S₄ and hetero-atomic groups have been removed from the solid layer. Thickness of the bis-carboxy DBIPTCA film after the drying stage was about 50 nm. After the annealing step, thickness of the layer decreased to about 70% of the initial thickness. This value was essentially reproducible in the above referenced temperature ranges and time.

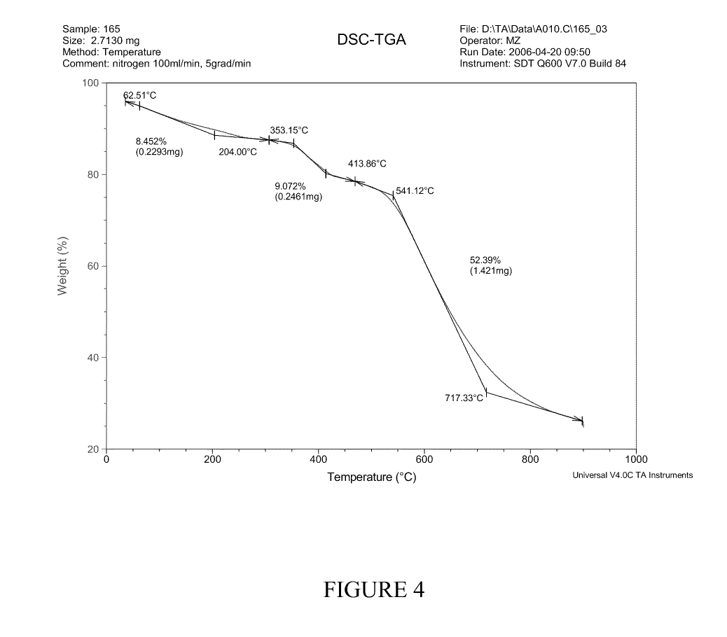

A thermo gravimetric analysis of the layer of bis-carboxy DBI PTCA is shown in FIG. 4. Thermal decomposition of bis-carboxy DBI PTCA has three main stages: 1) water and ammonia removal from the film (24-250° C.), 2) decarboxylation process (353-415° C.), and 3) DBI PTCA layer partial pyrolysis with carbon-conjugated residues forming (541-717° C.). The formula weight (FW) of Bis(carboxybenzimidazoles) of PTCA is shown in Table 6.

TABLE 6
Formula weight (FW) of Bis(carboxybenzimidazoles) of PTCA

| Structure | FW | Loss, % |
|---|---|---|
| 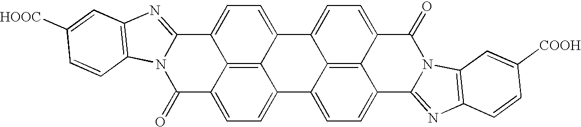 | 624.557 | 0 |
| 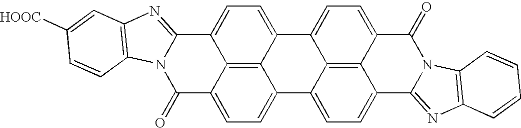 | 580.5475 | 7.05 |
| 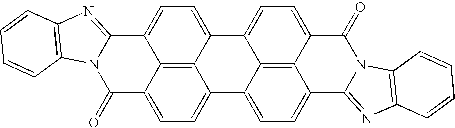 | 536.538 | 14.09 |
| 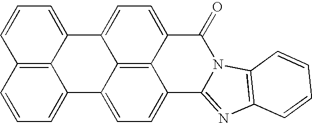 | 394.4236 | 36.85 |
| 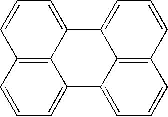 | 252.3093 | 59.60 |

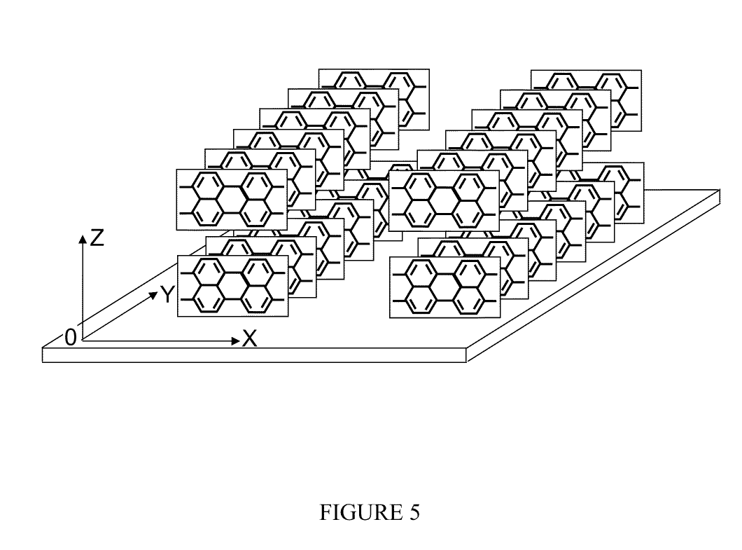

The resulting carbon-conjugated residues formed the intermediate anisotropic structure represented in FIG. 5.

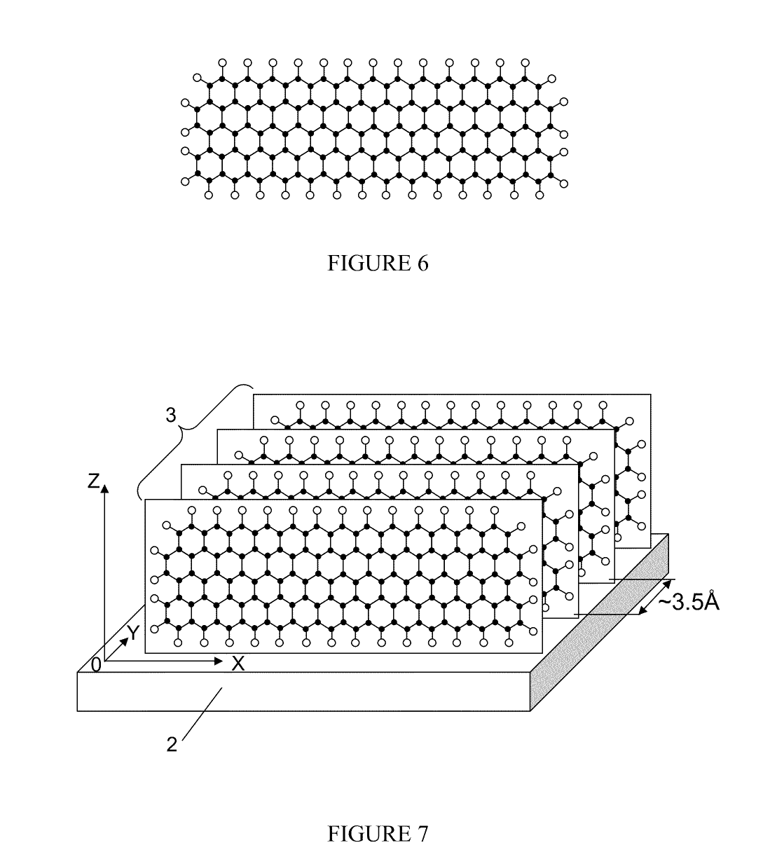

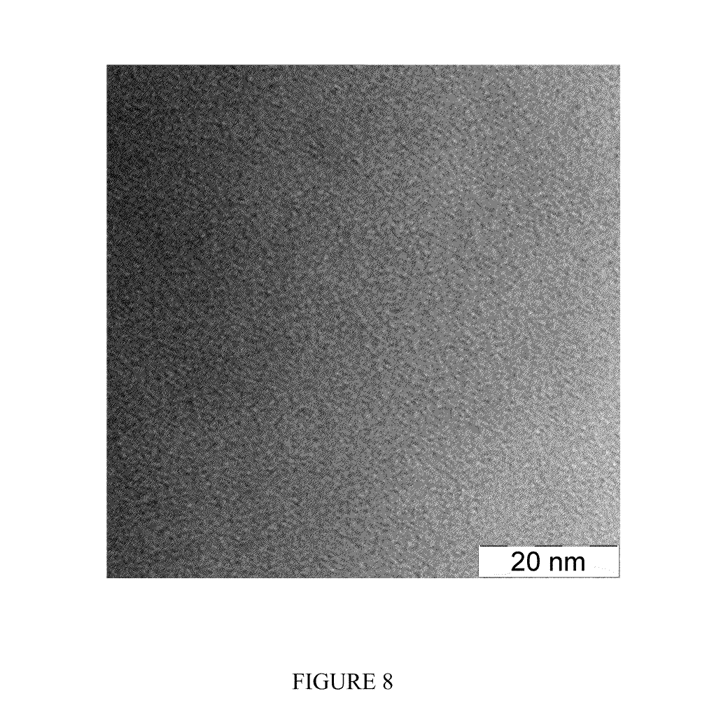

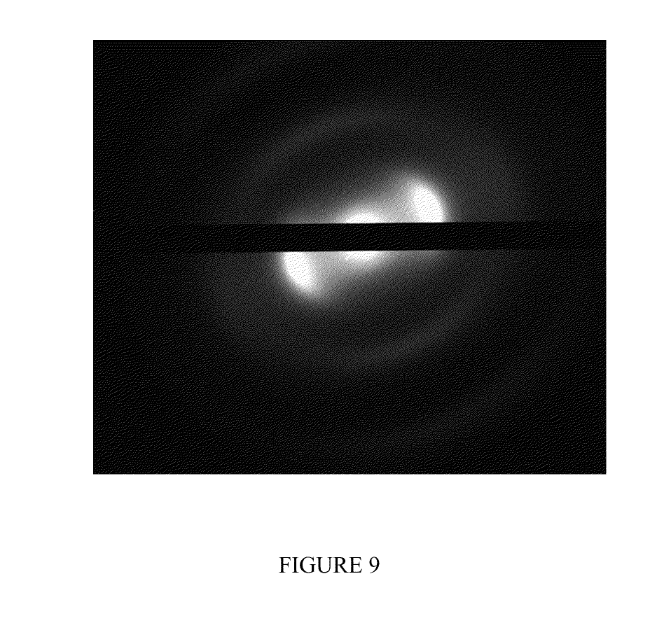

High-temperature annealing resulted in the formation of predominantly planar graphene-like carbon-based structures via fusion of the carbon-conjugated residues under high temperatures. One possible embodiment of such graphene-like carbon-based structures is shown schematically in FIG. 6. FIG. 7 shows schematically the anisotropic graphene-like ribtan layer (3) on the substrate (2) after the annealing step. TEM image of the ribtan layer formed on a substrate is shown in FIG. 8. There is global preferential orientation in the layer order. The orientation was also shown by electron diffraction images (FIG. 9). The diffraction image proves that the ribtan film have layered structure similar to α-graphite. There are two clear maxima related to 002 and 002 diffraction reflexes that correspond to 1D ordering in the layer in the direction perpendicular to graphene planes. The interplanar space is about 3.4 Å.

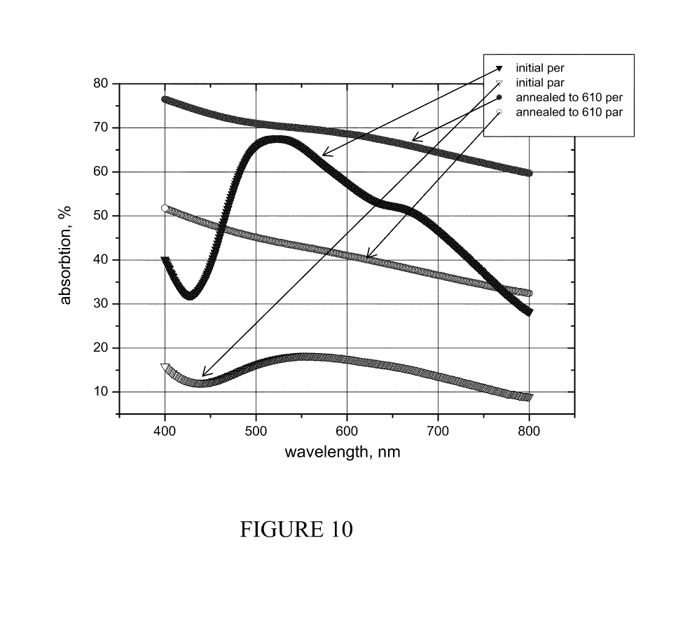

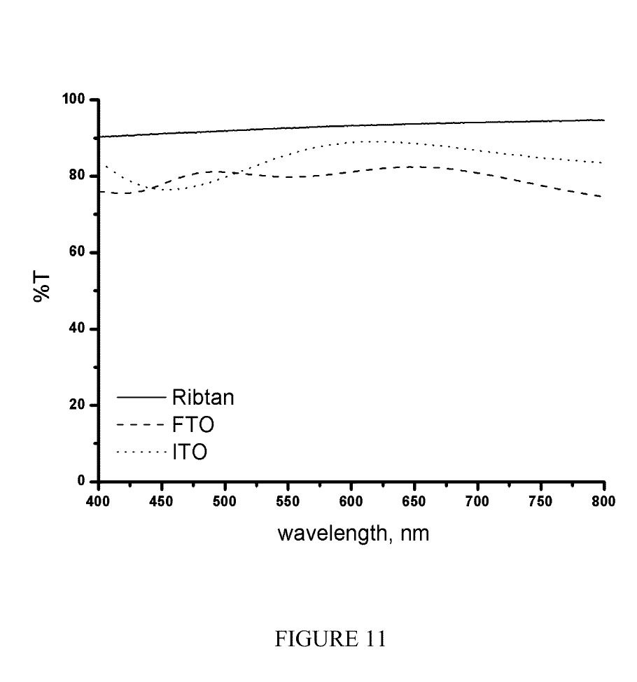

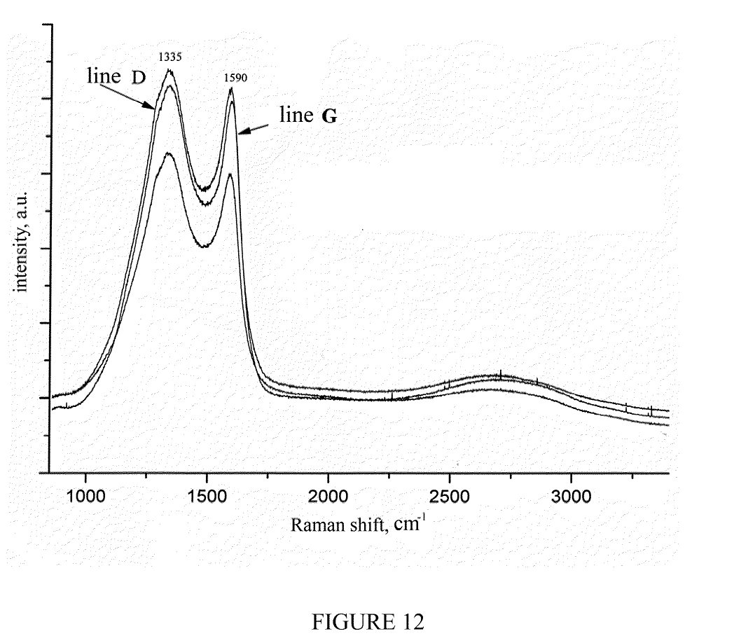

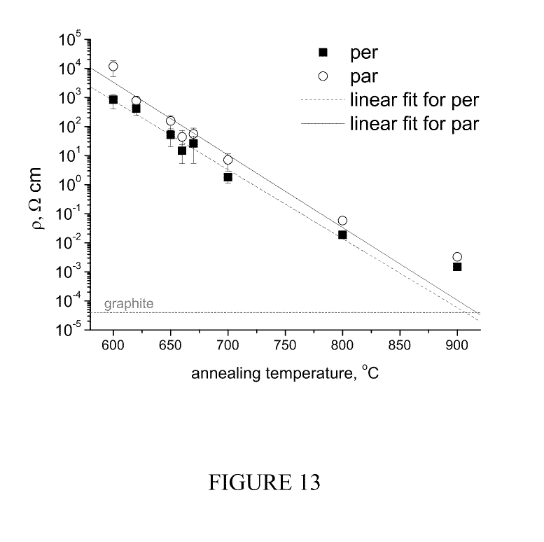

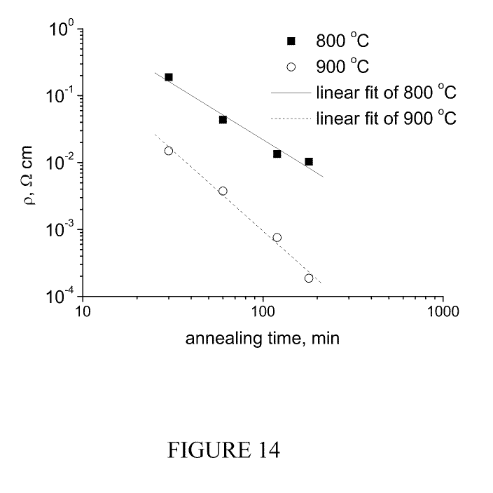

Absorption spectra of the annealed and dried layer of bis-carboxy DBI PTCA are shown in FIG. 10. The absorption spectrum of the annealed sample shows an optical anisotropy. Transmittance spectra of the annealed layer of bis-carboxy DBI PTCA are shown in FIG. 11. Transmittance spectra of the layers made of indium tin oxide (ITO) and fluorine tin oxide (FTO) are shown in FIG. 11 for comparison. FIG. 12 shows Raman spectra of the annealed samples. The spectra were taken at different points on the sample surface. The spectra include typical lines for sp² bonded carbon material. The position of these line G and its FWHM suggests that the ribtan layer consists of graphene layered structure. Line D is split which means that the surface of ribtan films consist of edges of graphene layers. Measurements of resistivity of the ribtan layers have been made using a standard 4-point probe technique. The resistivity of the ribtan layers was measured parallel (par) and perpendicular (per) to coating direction in order to detect electrical anisotropy of the films. Results of the measurements are shown in FIG. 13 and FIG. 14.

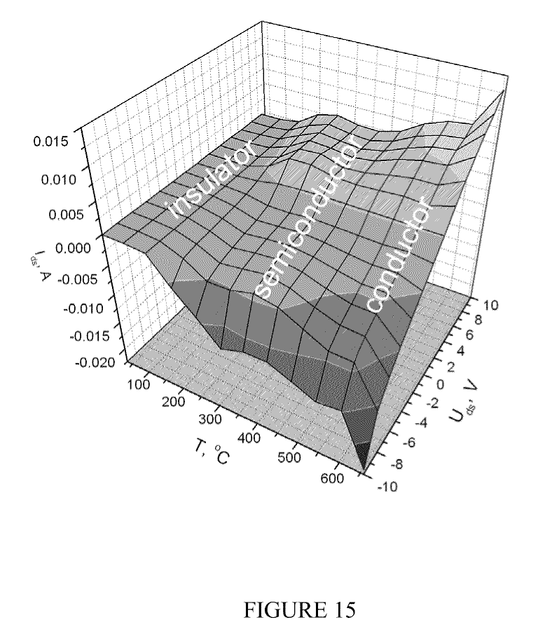

There is some anisotropy of resistivity. Resistivity along graphene ribbons (per) is lower than resistivity across the ribbons (par). The resistivity strongly depends on fusion temperature and exposure time. FIG. 13 shows resistivity as a function of maximum fusion temperature (Tₘₐₓ) and FIG. 14 shows resistivity as a function of time of sample exposure at maximum temperature. Generally, resistivity decreases with increasing of exposure time and fusion temperature. The resistivity perpendicular to the coating direction is about 2-3 times smaller than resistivity parallel to the coating direction. Thus, the ribtan layer possesses anisotropy of resistivity. Such anisotropy of the resistivity corresponds to a better charge transport in the direction along the graphene-like carbon-based structures. The voltage-current characteristics obtained at different annealing temperatures on bis-carboxy DBIPTCA layer are shown in FIG. 15. The ribtan layers are characterized by dependence of conductivity (a reciprocal value of electrical resistivity) on annealing temperature and by transition: insulating—semiconducting—conductor state. The high value of the measured conductivity proves the global (continuous) character of the ribtan layer.

### Example 12

The example describes the properties of the films based on optically transparent and electrically conductive layer based on a ribtan material that allows then serving as potential window electrodes for optoelectronics. FIG. 16 represents a two-layer (bilayer) organic photovoltaic cell in which the dissociation of excitons and the separation of bound charges proceed predominantly at the photovoltaic heterojunction. The organic photovoltaic device was based on the ribtan/bis-carboxy DBI PTCA/carboxy-CuPc/Al structure with Al top contact (4). Samples were coated on ribtan/glass substrate (layers 7 and 8). Top contact Al (4) was deposited by thermal evaporation. The copper-4,4′,4″,4′″-tetracarboxyphthalocyanine (carboxy-CuPc) is described by the following structural formula:

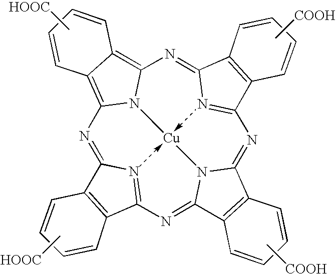

The bis-carboxy-DBI PTCA is described by the structural formula which is shown in FIG. 1.

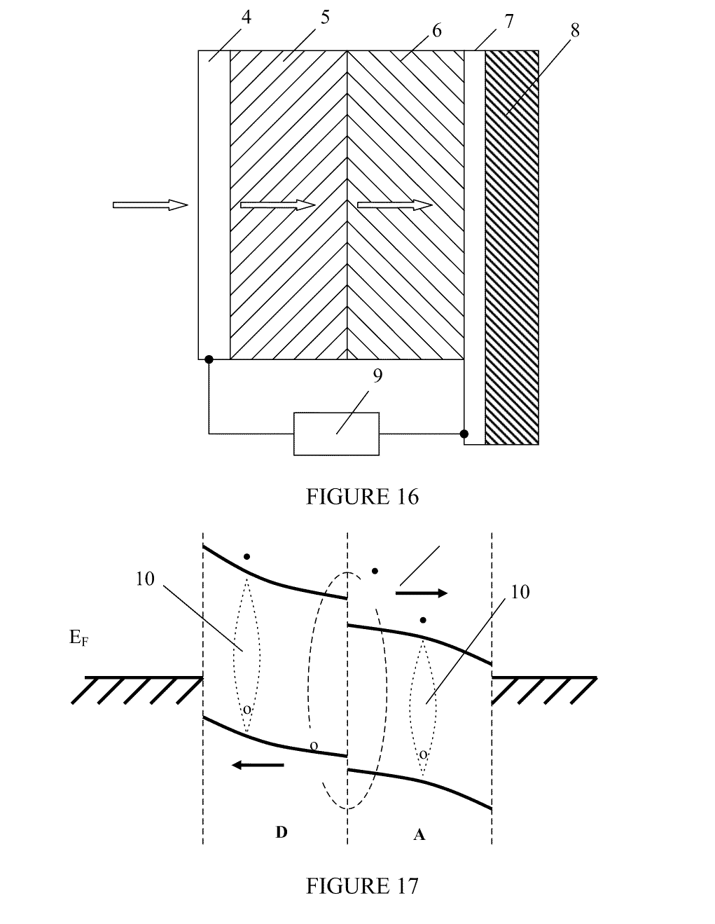

The built-in electric field is determined by the LUMO-HOMO energy difference between two materials forming the heterojunction. This device comprised two contacting photovoltaic layers—an electron donor layer (5) and an electron acceptor layer (6)—forming Ohmic contacts with the adjacent electrodes (4 and 7). The entire multilayer structure was formed on the substrate (8). The energy band diagram of this double-layer organic photovoltaic device is presented in FIG. 17. In this structure, bound electron—hole pairs (excitons 10) are generated by the incident electromagnetic radiation in both the electron donor (D) and acceptor (A) layers, with a photovoltaic heterojunction formed at the interface of these layers. This region features dissociation of excitons with the formation of mobile charge carriers, electrons and holes, moving toward the cathode and anode, respectively, under the action of the built-in electric field. The separated electrons and holes move to the corresponding electrodes in different layers, namely electrons drift from the heterojunction to the cathode via the electron acceptor layer, while holes drift from the heterojunction to the anode via the electron donor layer. This property of a double-layer organic photovoltaic structure reduces probability of the electron—hole recombination, thus increasing the photovoltaic conversion efficiency. Another advantage of the two-layer organic photovoltaic device over the single layer counterpart is the basic possibility of using a wider wavelength range of the incident radiation. The electron donor and acceptor layers have to be made of materials possessing different absorption bands.

Good efficiency was achieved on structure, wherein the first layer thickness was equal to 70 nm and second layer thickness was equal to 120 nm. Decreasing of thickness of the following layers was complicated by the decreased layer quality related to the thickness decreasing and possibility of shorts.

### Example 13

The example describes synthesis of an organic donor-bridge-acceptor (DBA) material which is used as active layer of the solar cell described in Example 14. Synthesis contains the following stages:

### 1. Synthesis of 3,4-Bis(hexadecyloxy)benzonitrile

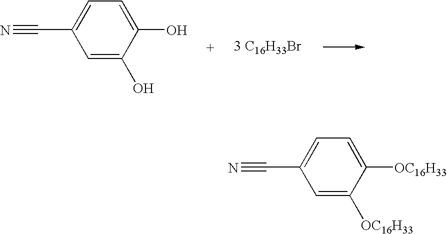

3,4-Dihydroxybenzonitrile (10.0 g), potassium carbonate (61.4 g), 1-bromohexadecane (56.5 mL) and potassium iodide (0.1 g) were mixed with dimethylformamide (200 mL). After that the reaction mass was agitated at 108-112° C. for 48 hours. Then the reaction mass was filtered in a hot state. The white precipitate was suspended in chloroform (300 mL) and the suspension was stirred with heating for 10-15 minutes and then filtered. The filtrates were combined and heated till the solution was formed. The hot solution was washed with warm water (3×300 mL). The obtained organic fraction was dried under sodium sulfate for an hour. Then the solution was filtered off and evaporated from solvent under vacuum with a water-jet pump. The white solid was dried with adding toluene (150 mL) in three steps. The crude product (50.3 g) was the dissolved in chloroform (˜50 mL) and the resulted solution was filtered through a silica gel chromatographic column (eluent—Chloroform, l=150 mm, Ø70 mm). The solvent was evaporated under vacuum with a water-jet pump, and the solid was recrystallized from acetone (390 mL) and dried at 40° C. in an oven. Yield 37.4 g.

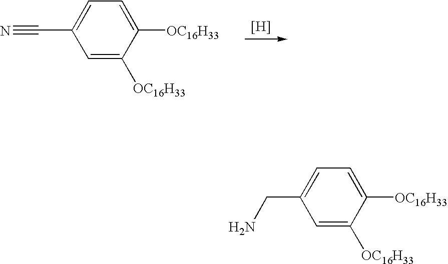

### 2. Synthesis of 3,4-Bis(hexadecyloxy)benzylamine

A warm solution of 3,4-bis(hexadecyloxy)benzonitrile (17.0) in absolute tetrahydrofuran (140 mL) was added to a suspension of lithium aluminum hydride (4.8 g) in absolute tetrahydrofuran (80 mL) dropwise with stirring for 15 minutes at 20-40° C. under argon atmosphere. The resulted reaction mixture was refluxed for 2 hours and cooled to room temperature and left overnight.

Then the reaction mixture was added dropwise into ice (˜800 g). The resulted white slurry was mixed with 20% aqueous solution of sodium hydroxide (1000 mL), warmed and stirred at 40° C. for 30 min. Obtained mixture was extracted with warm chloroform (3×400 mL), the combined extract was washed with water (2×300 mL) and dried over sodium sulfate. Solvent was evaporated on a rotary evaporator. Yield 16.6 g.

### 3. Synthesis of 1,7-dibrom-3,4,9,10-perylenetetracarboxylic acid dianhydride

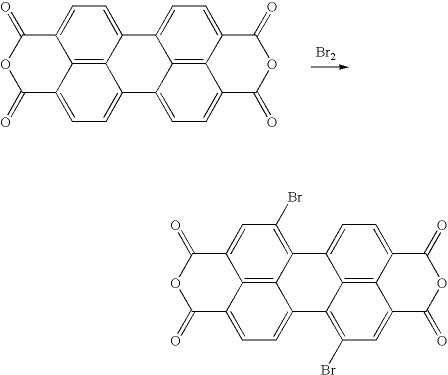

3,4,9,10-perylenetetracarboxylic acid dianhydride (50 g) was charged into 100% sulfuric acid (423 mL) at room temperature (22-24° C.). The resultant mixture was left overnight with stirring at the same temperature.

Iodine (1.19 g) was added to the reaction mixture. After that it was heated up to 85-87° C. Bromine (14.2 mL) was added dropwise to the hot reaction mixture for 7 hours. The reaction mass was agitated at 85° C. for 1 hour more, cooled down to 50° C. and excess of bromine and BrI was removed under reduced pressure with using of a water-jet.

The solution was left overnight. Then water (127 mL) was added dropwise to the violet solution for an hour with efficient stirring. The obtained suspension was cooled to 35° C., and the red precipitate was filtered off. The precipitate was rinsed with 86% sulfuric acid (600 mL), suspended in water (2×750 mL), filtered off and washed with water till neutral pH value and discoloration of the washing water. The filter cake was dried at 75° C. for 4-5 hours. Yield was 54.2 g.

### 4. Synthesis of N-(3,4-bis(3,4-bis(hexadecyloxy)benzyl)-1,7-dibromo-3,4:9,10-tetracarboxylmide anhydride

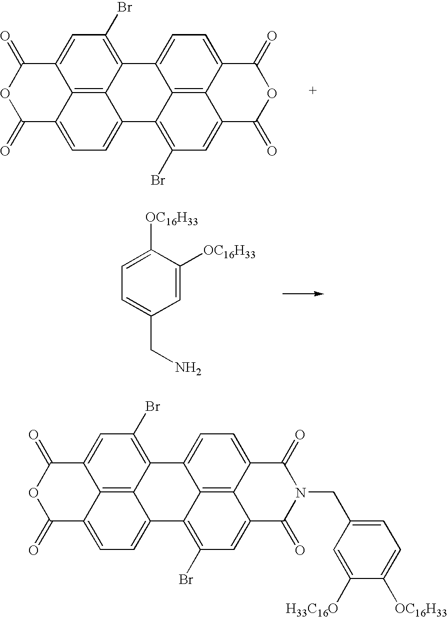

1,7-dibromo-3,4,9,10-perylenetetracarboxylic acid dianhydride (1.55 g) and benzoic acid (10 mg) were charged into 3-chlorophenol (10 mL). The resultant mass was degassed and filled with argon 3 times at 80° C. (bath temperature) and heated at 150° C. for 1 h, then cooled to 75°.

A solution of 3,4-bis-(hexadecyloxy)benzylamine (2.00 g) in 3-chlorophenol (2 mL) was added. The flask was evacuated and filled with argon 3 times, and temperature of the bath was rose to 140° C. for 50 minutes, then several drops of 3-chlorophenol were distilled off at bath temperature 135° C. for 10 minutes.

The reaction solution was added to hot methanol (100 mL) and precipitate was filtered washed with methanol (100 mL). Filter cake was re-precipitated form the mixture of methanol-chloroform (100 mL/5 mL) and dissolved in the mixture of toluene (200 mL) and acetic acid (30 mL). After that the solution was boiled for 15 minutes reducing volume to ½ of initial volume then evaporated to dryness on a rotary evaporator.

Residue was dissolved in eluent mixture (silica gel, d=4.8, l=24 cm, eluent chloroform-petroleum ether-methanol-Ethylacetate-100:6:0.3:0.5). Yield 1.05 g.

### 5. Synthesis of N-(3,4-bis(3,4-bis(hexadecyloxy)benzyl)-1,7-diphenoxy-3,4:9,10-tetracarboxylmide anhydride

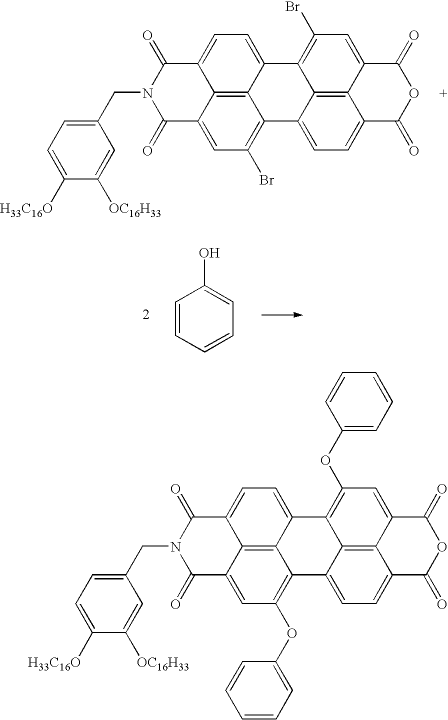

The mixture of N-(3,4-bis(3,4-bis(hexadecyloxy)benzyl)-1,7-dibromo-3,4:9,10-tetracarboxylmide anhydride (1.00 g), phenol (0.250 g) and Cesium carbonate (0.360 g) was evacuated and filled with argon several times, then dimethylformamide was added (13 mL) and resultant mixture was evacuated and filled with argon 3 times more. Reaction mass was heated up to 135° C. and agitated at 130-135° for 45 minutes. Two portions of dimethylformamide (2 mL each) were added during heating.

Reaction mixture was diluted with 5% hydrochloric acid (200 mL) and stirred overnight. Precipitate was filtered, dissolved in chloroform (200 mL), washed with 7% hydrochloric acid (200 mL), organic layer was dried over sodium sulfate, filtered, evaporated, and precipitated from the mixture of chloroform-methanol. Red-violet precipitate was dried and separated using silica gel column (l=24, d=4.8 cm, eluent chloroform-petroleum ether-methanol-ethylacetate-100:10:0.3:1.0). Yield 280 mg.

### 6. Synthesis of 4′-nitrobiphenyl-4-carbaldehyde

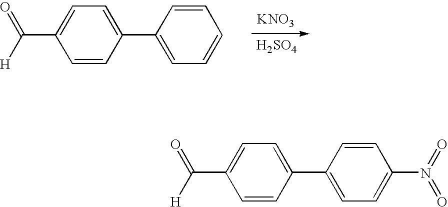

4-Biphenylcarboxaldehyde (10.2 g) was dissolved in concentrated sulfuric acid (225 ml) at the room temperature with stirring. Then reaction mass was cooled down to ˜0° C. After that Potassium nitrate (5.7 g) was charged into reaction mass by parts at ˜0° C. Then reaction mass was agitated for 2.5 hours at ˜0° C. After that the reaction mass was poured onto ice (1 kg). After ice melted the precipitate was filtered and rinsed with water. Filter cake was suspended in water (500 ml). Precipitate was filtered and rinsed with water. Wet filter cake was dissolved in the boiling ethanol (200 ml). After self cooling the precipitate was filtered, rinsed with water and dried in vacuum at ˜50° C. Yield 6.68 g.

### 7. Synthesis of 5,10,15,20-Tetrakis(4′-nitrobiphen-4-yl)porphyrin

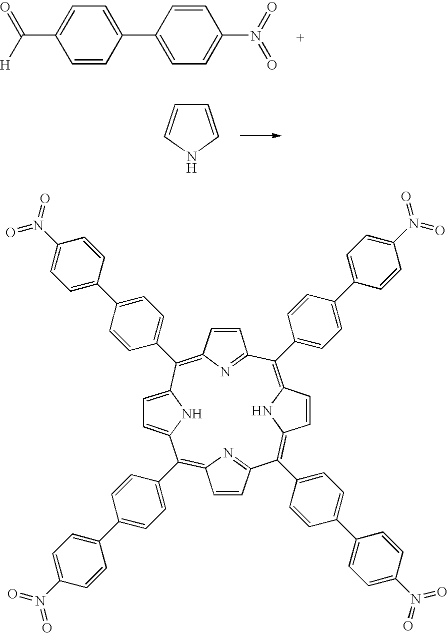

Mixture of 4′-nitrobiphenyl-4-carbaldehyde (3.26 g), propionic acid (65 ml) and acetic anhydride (2.17 ml) was heated up to boiling. After that pyrrole (0.99 ml) was charged by portions into the reaction mass. Then reaction mass was boiled for 2 hours with agitation. After self cooling the precipitate was filtered, rinsed with methanol and water and dried at ˜100° C. Obtained compound was agitated in the boiling pyridine (20 ml) for 2 hours. After self cooling, the precipitate was filtered, rinsed with methanol and water and dried at ˜100° C. Yield 1.27 g.

### 8. Synthesis of 5,10,15,20-Tetrakis(4-aminobiphenyl)porphyrin

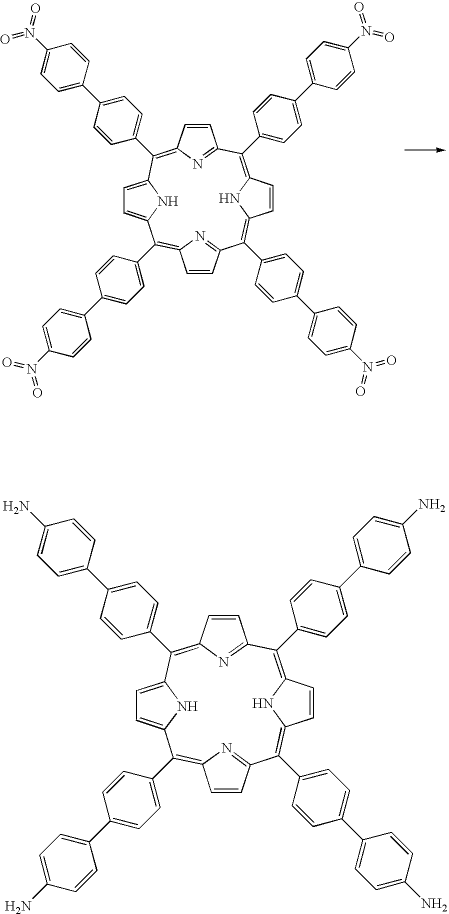

Mixture of 5,10,15,20-tetrakis(4′-nitrobiphen-4-yl)porphyrin (1.27 g), tih(II) chloride dihydrate (5.8 g) and concentrated hydrochloric acid (81 ml) was agitated at ˜80° C. for 2 hours. After self cooling the reaction mass was diluted with water (81 ml). Precipitate was filtered and rinsed with 6N hydrochloric acid (50 ml). Filter cake was agitated in warm water (˜60° C.) for 30 minutes. After self cooling the suspension was neutralized with concentrated ammonia solution. Precipitate was filtered, rinsed with water and dried at ˜100° C. Obtained compound was purified by chromatography on the filter with aluminum oxide (l=40 mm, d=70 mm) and dimethylformamide as eluent. Solution was concentrated on the rotary evaporator and diluted with water. Precipitate was filtered, rinsed with water and dried at ˜100° C. Yield 380 mg.

### 9. Synthesis of donor-bridge-acceptor (DBA)-molecule

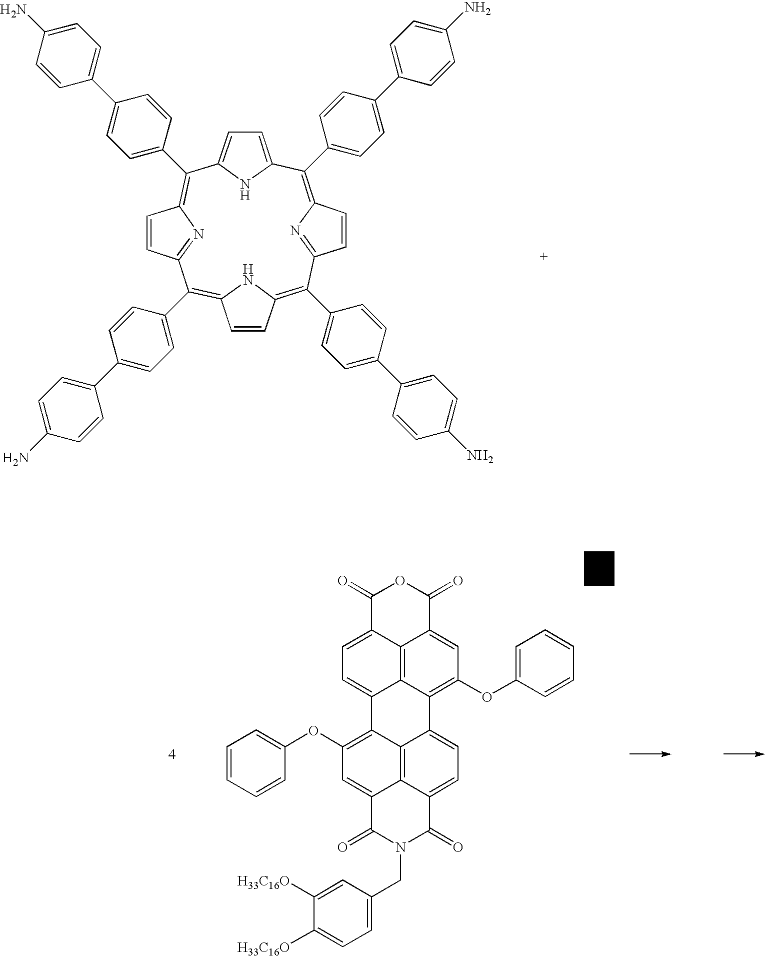

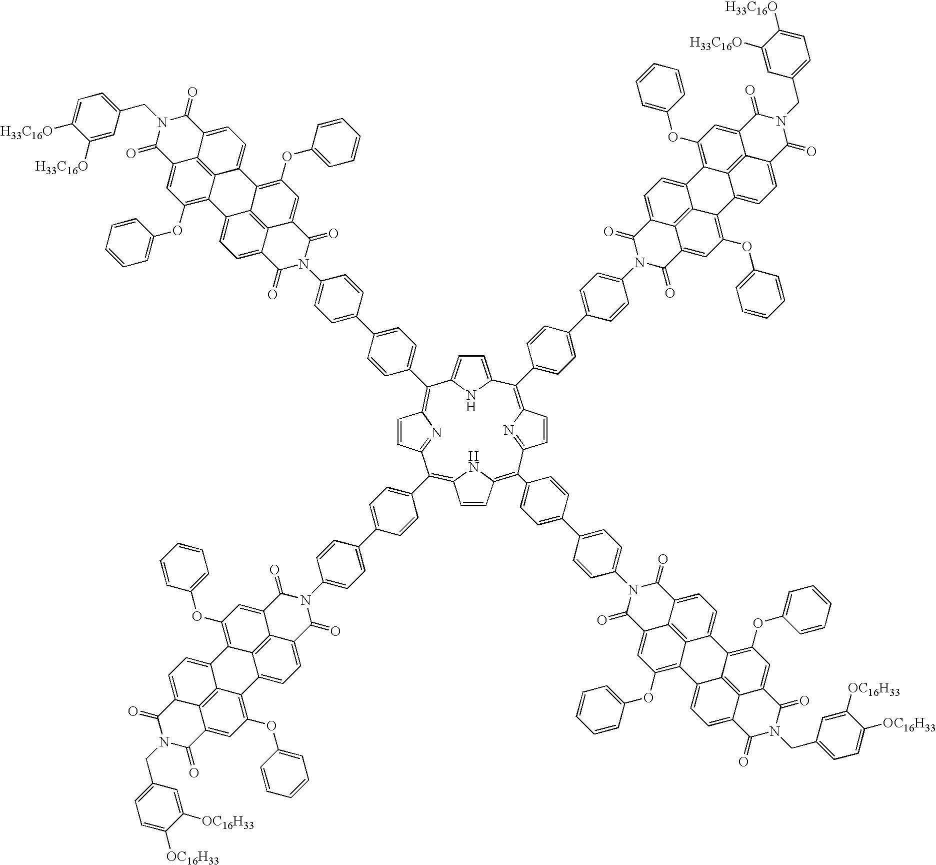

Mixture of N-(3,4-bis(3,4-bis(hexadecyloxy)benzyl)-1,7-diphenoxy-3,4:9,10-tetracarboxyimide anhydride (0.140 g), 5,10,15,20-Tetrakis(4-aminobiphenyl)porphyrin (0.020 g), benzoic acid (2 mg) and 3-chlorophenol (1.0 mL) was degassed at 80° C., and heating with stirring was continued for 2 days at 150° C.

Warm methanol (50 mL) was charged into the reaction mixture. The solvent was removed on a rotary evaporator. Thin film of organic material was washed with mixture of Diethyl ether and ethanol (2×150 mL:30 mL) and then passed through a alumina column (18×3 cm, eluent chloroform-methanol (20:1)).

Red fraction was collected, filtered through a PALL glass fiber filter and evaporated to dryness. Residue was washed with Diethyl ether and ethanol (200 mL:20 mL), dried. Yield 70 mg.

### Example 14

The example describes prototypes of solar cell with the ribtan electrode. The solar cell was based on organic donor-bridge-acceptor (DBA) material, synthesis of which is described in Example 13. As a first step, a water solution of disulfo violanthrone and disulfo isoviolanthrone was applied on quartz substrates using Mayer rod. The predominantly planar carbon-conjugated cores of the molecules are shown in Table 2, structures 24 and 25 respectively. The sulfo groups provided solubility in water. Then the obtained samples were annealed in nitrogen flow at 800° C. during 1 hour. The obtained ribtan coatings were relatively thick with optical transitions about 40% and surface resistance about 10 kΩ/sq. Toluene solution of DBA material was coated on obtained ribtan films using Mayer rod technique. After that, thin semitransparent layer of Al was deposited on the top of the layer of DBA material by thermal evaporation in vacuum.

Current-voltage characteristics of the samples was measured under illumination with lamp (light characteristics) and in black box (dark characteristics) using Keithley 2440. The illumination was performed through Al semi-transparent layer. The surface area of the prototypes was about 6 cm². The results of the prototypes testing are shown in the Table 7.

TABLE 7
Results of testing of solar cells with ribtan electrodes

| | Sample | Ribtan material | I_{sc}, μA | V_{oc}, V |
|---|---|---|---|---|
| 1 | quartz/ribtan/DBA/A1 | Structure 24 in | 1.5 | 0.75 |
| | | Table 2 | | |
| 2 | quartz/ribtan/DBA/A1 | Structure 25 in | 6.5 | 1 |
| | | Table 2 | | |

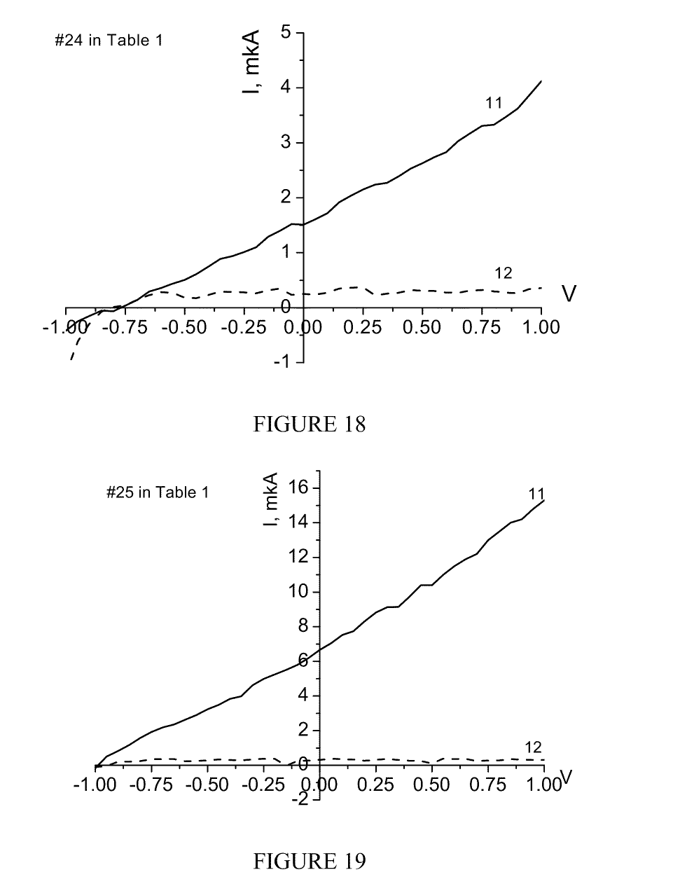

The samples demonstrated high photovoltages without any selective layers. Ribtan in the samples conducted holes. Current-voltage characteristics of the samples with ribtan made of sulfo derivates of molecules shown in the Table 2, structures 24 and 25 are shown in the FIGS. 18 and 19 respectively, wherein 11 is light characteristic and 12 is dark characteristic.

### Example 15

This example describes a low-temperature method of producing a film comprising at least one optically transparent and electrically conductive layer based on a ribtan material according to the present invention. The film comprises a ribtan layer located on a substrate.

The ribtan layer comprising graphene-like carbon-based structures was formed with a mixture of bis(carboxybenzimidazoles) of prerylenetetracarboxylic acids (bis-carboxy DBIPTCA). As a first step, a water solution of bis-carboxy DBIPTCA was applied on a substrate. The solution comprised a mixture of six isomers as shown in FIG. 1, which predominantly planar carbon-conjugated cores are shown in Table 1, structures 4 and 5. Bis-carboxy DBIPTCA is a π-conjugated organic compound, where the predominantly planar carbon-conjugated core (CC in formula I) comprises rylene fragments, the benzimidazole groups serve as hetero-atomic groups, and carboxylic groups serve as substituents providing solubility. The molecular structure provides for the formation of rod-like molecular stacks.

For the purpose of this example glass was used as a substrate material. The Mayer rod technique was used to coat the water-based solution of bis-carboxy DBIPTCA. During the second step drying is performed. By the end of the drying step, the layer usually retains about 10% of the solvent. As a result of the drying step the layer comprised rod-like supramolecules oriented along the coating direction. FIG. 2 schematically shows the supramolecule (1) oriented along the y-axis and located on the substrate (2). The distance between the planes of bis-carboxy DBIPTCA is approximately equal to 3.4 A.

During the next step the solid layer was placed into a gas-phase environment containing molecules which are sources of free radicals or benzyne fragments. In this example azobenzene C₆H₅N₂C₆H₅ was used as a source of free benzene radicals in a gas phase.

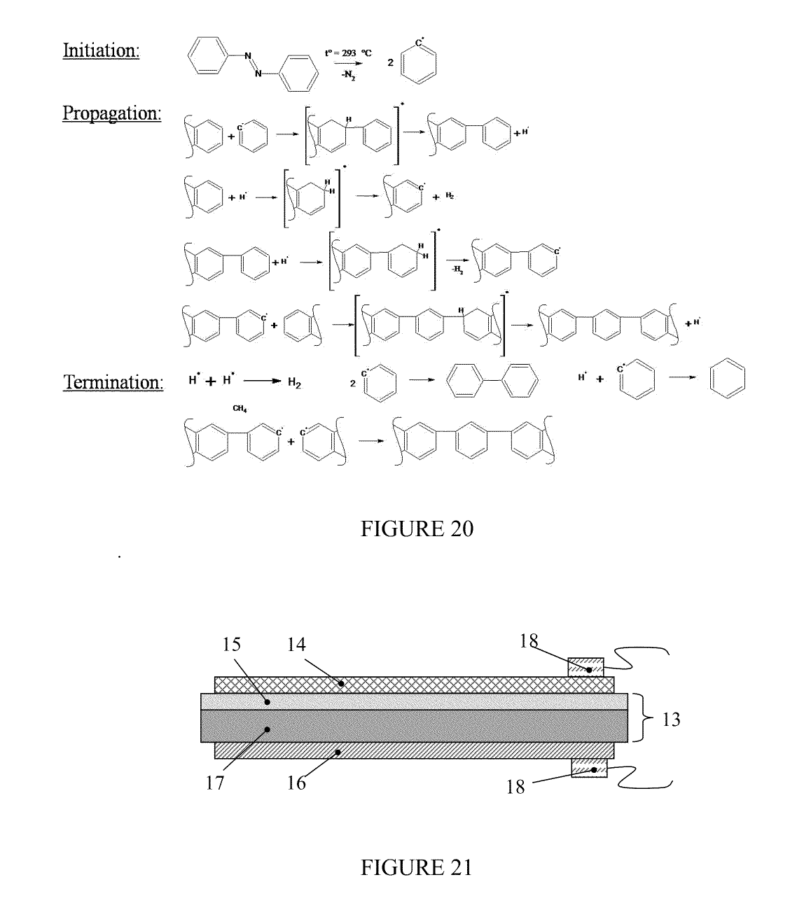

Heating up to 300° C. was used for evaporation of azobenzene and formation of benzene free radicals. The chemical reactions taken place in the reactor are schematically shown in FIG. 20. Radical induced polymerization occurred. The process of the radical polymerization consists of three main steps which are initiation step, propagation step and termination step.

The initiation step was decomposition of azobenzene and free benzene radicals were developed. The reaction is thermally activated and temperature of the azobenzene decomposition was not higher than 300 degrees C.

The free benzene radicals reacted with polyaromatic precursor molecules in the solid layer via substitution reaction: one hydrogen atom of polyaromatic core was substituted by one benzene ring, through a homolytic pathway. The reaction leads to closing of gaps with benzene between aligned discotic precursor molecules in a solid precursor layer and formation of free hydrogen radicals. The resulting free hydrogen radical reacts with carbon conjugated cores and cause formation of free radicals on polyaromatic cores of precursor molecules. Due to global alignment of rod-like supramolecules in the solid precursor layer and addition of benzene radicals to the polyaromatic cores the neighbour discotic molecules with formed free radicals on their edges were ready for the joint reaction. Hence conjugation of the precursor molecules with covalent Csp²-Csp² bonds into graphene-like carbon-based structures was propagated in the ribtan film and formation of the ribtan layer took place at low temperature (not much higher than 300° C.). The free radicals can annihilate with each other and disappear from the reaction during termination step.

### Example 16

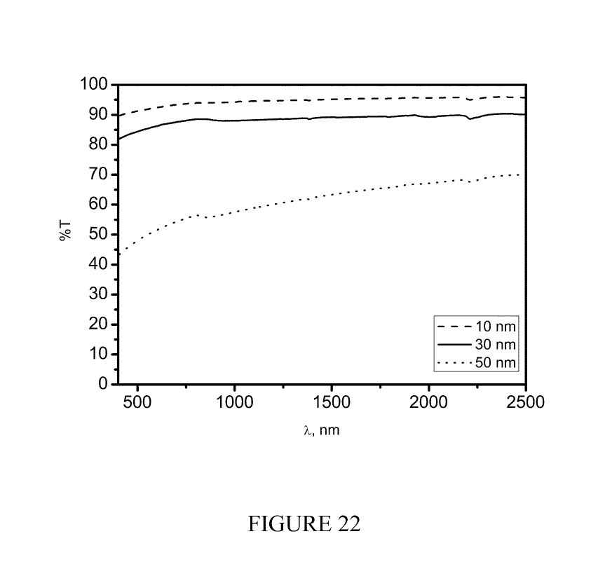

The Example describes a silicon solar cell with the transparent ribtan electrode. Silicon wafer with preliminary built p-n junction 13 was used as a substrate for the ribtan layer. Formation of the transparent ribtan layer 14 was performed as described in Example 11 on the top of the wafer (above n-doped Si layer 15). The transparent ribtan layer 14 was used as a transparent electrode. Al layer 16 was deposited by thermal evaporation in vacuum on the other side of the sample (above p-doped Si region 17). Indium contacts 18 were used for connection to the sample. The resulting multilayer structure is shown in FIG. 21.

Testing of the photovoltaic properties of the prepared sample was done under illumination of sun light simulator with power 1 kW/m². Under illumination the device generated electrical current about 2 mA and voltage about 0.4 V.

### Example 17

The present example describes an optical transmittance of ribtan films in UV, visible and near IR regions of optical spectrum. The conductive ribtan layer was formed on quartz substrates as described in the Example 11. Ribtan films with thicknesses about 10, 30 and 50 nm were prepared. The optical transmission spectra of the obtained ribtan layers were measured using UV-Vis-NIR spectrophotometer Lambda 950 (Perkin-Elmer, USA). The resulted spectra are shown in the FIG. 22. Transmittance of the ribtan films is strongly dependant on thickness of the ribtan layers. Thin ribtan films have high transmittance in UV, visible and near IR regions of optical spectrum.

### Example 18

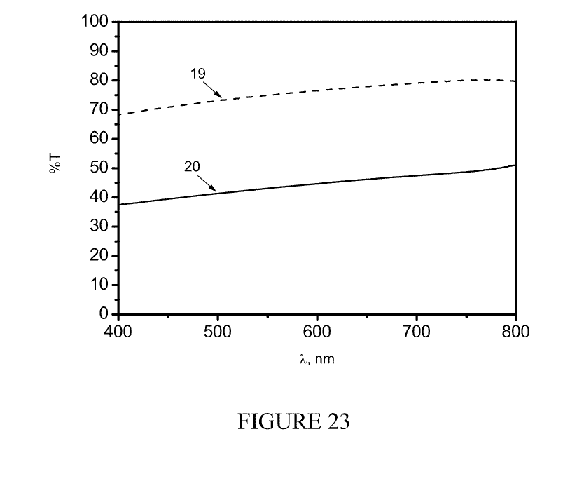

The present example illustrates polarizing properties of the ribtan layer. As a first step the ribtan layer on a quartz substrate was made as described in the Example 11 at 800 degrees C. during 2 hours. Thickness of the ribtan layer was about 50 nm. Optical transition spectra of the layer shown in FIG. 23 was measured in two perpendicular light polarization—parallel to coating direction (19) and perpendicular to coating direction (20). Transmittance spectrum of a clean quartz plate was used as a baseline.

We calculated average dichroic ratio Kd from the obtained spectra using the following equation

$$Kd = \frac{log {T}_{per}}{log {T}_{par}} ,$$

where Tₚₑᵣ and Tₚₐᵣ are the measured optical transmission of the ribtan film coating direction parallel and perpendicular to the polarizing axis of a referenced polarizer. Kd was 2.9 for the examined ribtan film.

Although the present invention has been described in detail with reference to a particular preferred embodiment, persons possessing ordinary skill in the art to which this invention pertains will appreciate that various modifications and enhancements may be made without departing from the spirit and scope of the claims that follow.

What is claimed is:

1. A film comprising at least one optically transparent and electrically conductive layer based on a ribtan material.

2. A film according to claim 1, comprising two or more optically transparent and electrically conductive layers, wherein at least two said layers are based on different ribtan materials.

3. A film according to claim 1, wherein at least one optically transparent and electrically conductive layer is transparent in the UV, visible and near IR regions of optical spectrum.

4. A film according to claim 1, wherein at least one optically transparent and electrically conductive layer possesses polarizing properties in the visible spectral range.

5. A film according to claim 1, wherein at least one optically transparent and electrically conductive layer has an optical transparency of at least 80% for 550 nm light and a resistivity of less than 0.002-0.029 Ohm·cm.

6. A film according to claim 1, further comprising a substrate, wherein the substrate is made of a flexible or a rigid material, and wherein the surface of the substrate is flat, convex, concave, or any combination thereof.

7. A film according to claim 6, wherein the substrate is made of one or several materials of the group comprising Si, Ge, SiGe, GaAs, diamond, quartz, silicon carbide, indium arsenide, indium phosphide, silicon germanium carbide, gallium arsenic phosphide, gallium indium phosphide, plastics, glasses, ceramics, metal-ceramic composites, metals, and comprises doped regions, circuit elements, and multilevel interconnects, and wherein said plastic material is selected from the group comprising polycarbonate, Mylar, polyethylene terephthalate (PET) and polyimide.

8. A film according to claim 6, wherein the substrate is transparent for electromagnetic radiation selected from the list comprising the UV, visible and near IR regions of optical spectrum.

9. A film according to claim 1, further comprising a transparent adhesive layer.

10. A film according to claim 9, further comprising a protective coating on top of the transparent adhesive layer.

11. A film according to claim 1, wherein the ribtan material is prepared using a solution comprising at least one π-conjugated organic compound of the general structural formula I or a combination of organic compounds of the general structural formula I: 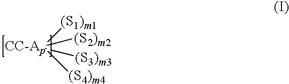
where CC is a predominantly planar carbon-conjugated core;
A is an hetero-atomic group;
p is 0, 1, 2, 3, 4, 5, 6, 7, or 8;
S₁, S₂, S₃, and S₄ are substituents;
m1, m2, m3 and m4 are 0, 1, 2, 3, 4, 5, 6, 7, or 8; and
sum (m1+m2+m3+m4) is 1, 2, 3, 4, 5, 6, 7, 8, 9 or 10.

12. A film according to claim 11, wherein said organic compound comprises one or more rylene fragments, and wherein said organic compound comprising rylene fragments has a general structural formula selected from the group comprising structures 1-23:

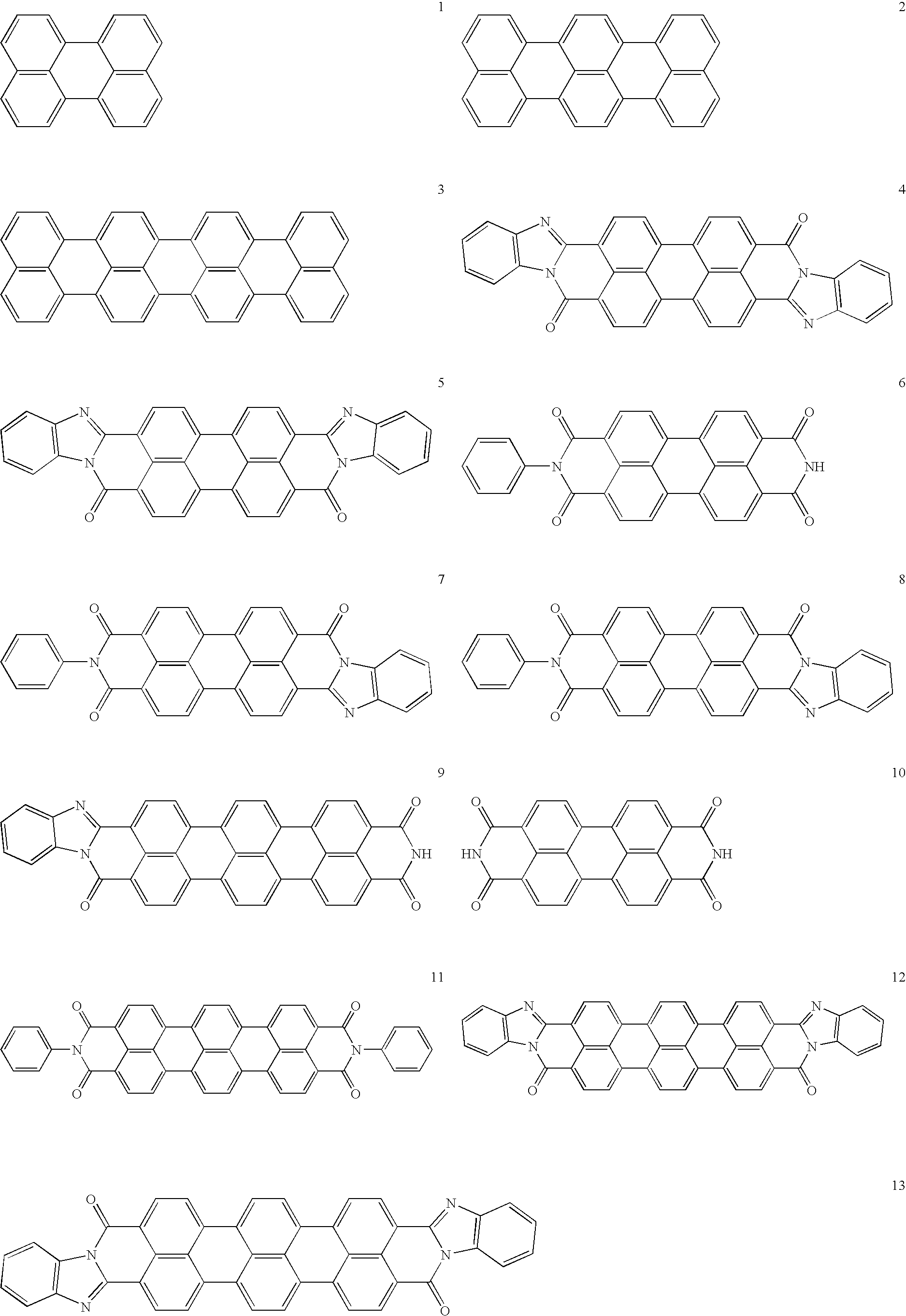

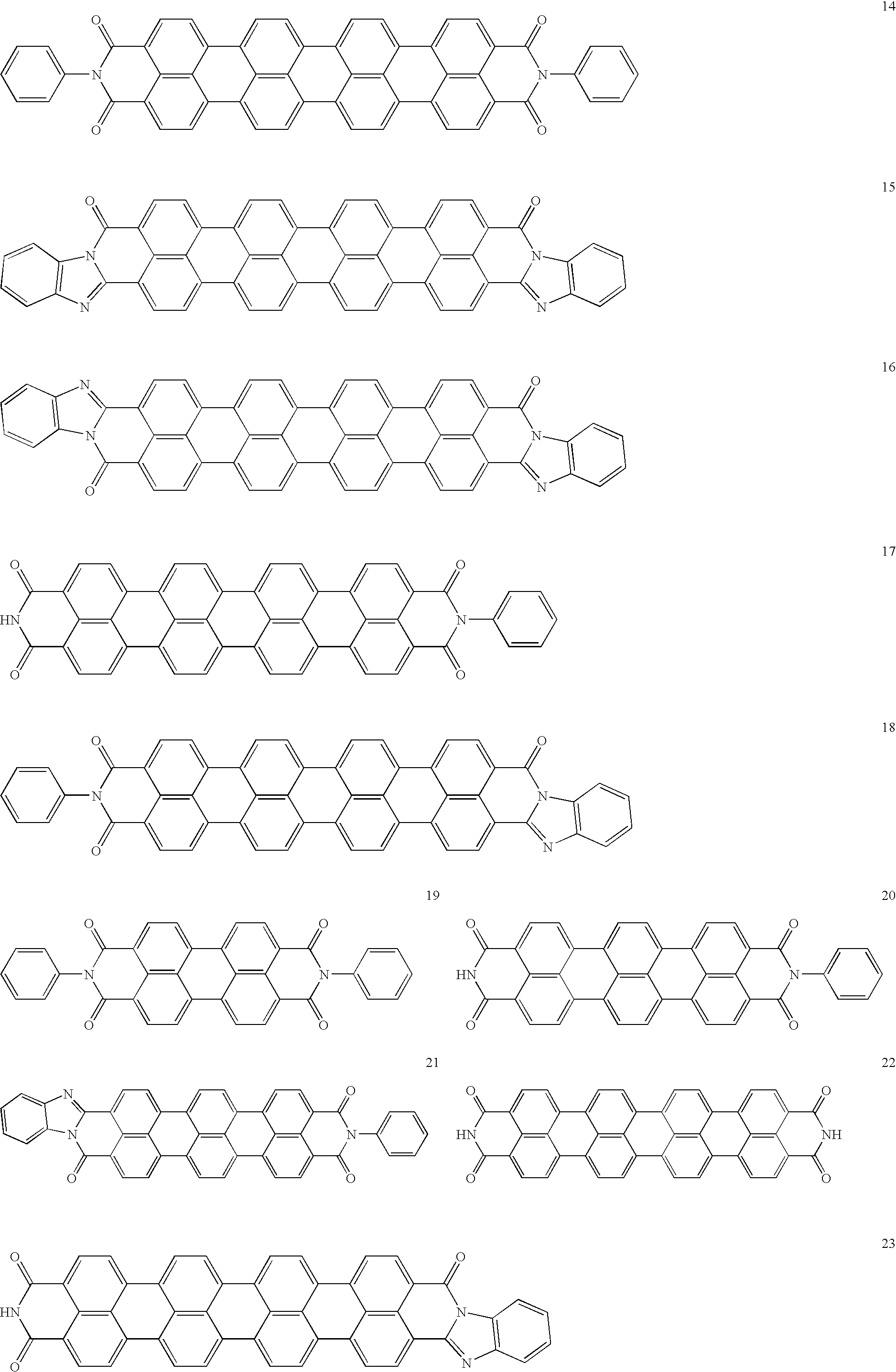

13. A film according to claim 11, wherein said organic compound comprises one or more anthrone fragments, and wherein said organic compound comprising anthrone fragments has a general structural formula selected from the group comprising structures 24-31:

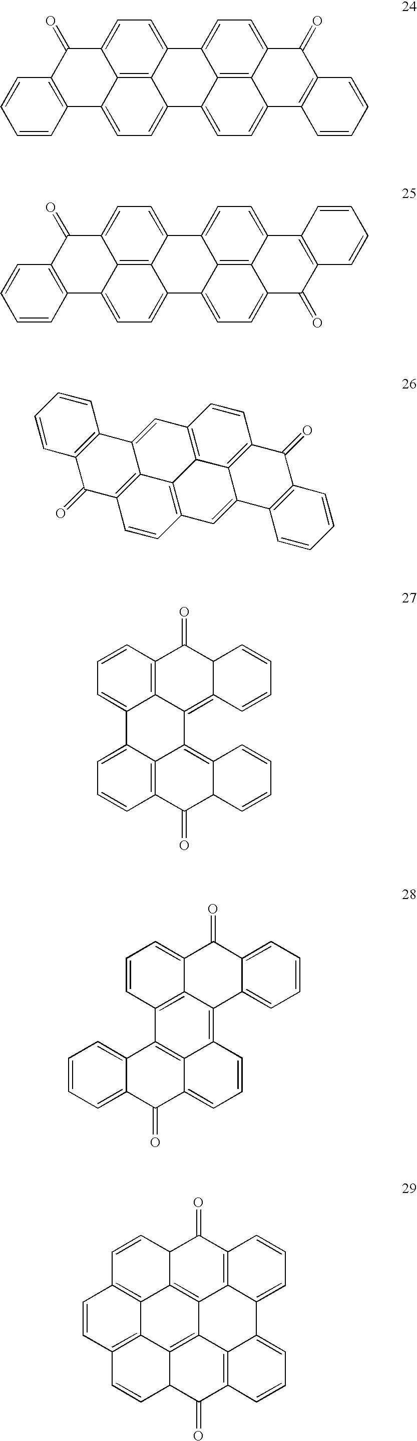

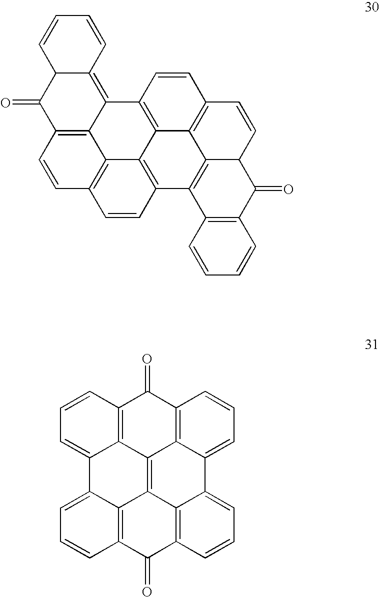

14. A film according to claim 11, wherein said organic compound comprises fused polycyclic hydrocarbons, and wherein said organic compound comprising fused polycyclic hydrocarbons selected from the list comprising truxene, decacyclene, antanthrene, hexabenzotriphenylene, 1.2,3.4,5.6,7,8-tetra-(peri-naphthylene)-anthracene, dibenzoctacene, tetrabenzoheptacene, peropyrene, hexabenzocoronene, violanthrene, isoviolanthrene has a general structural formula from the group comprising structures 32-43.

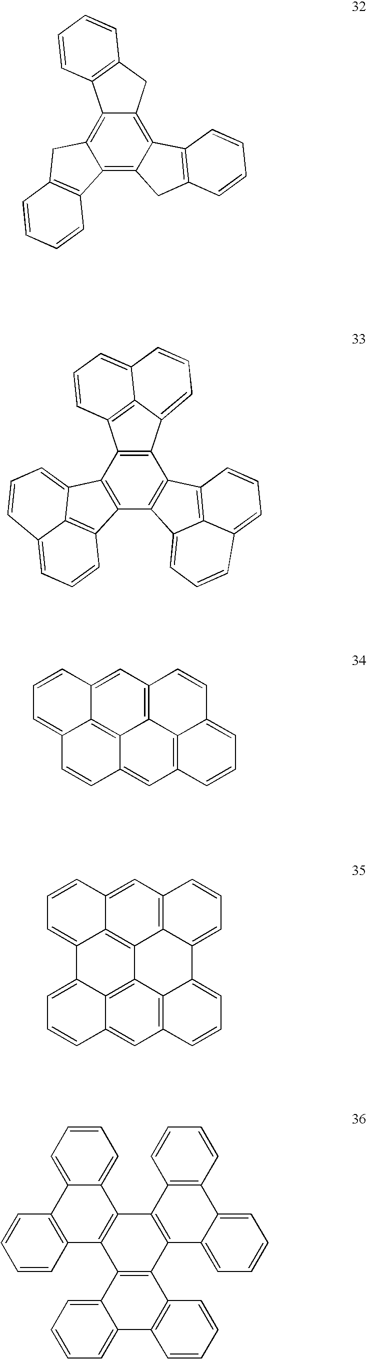

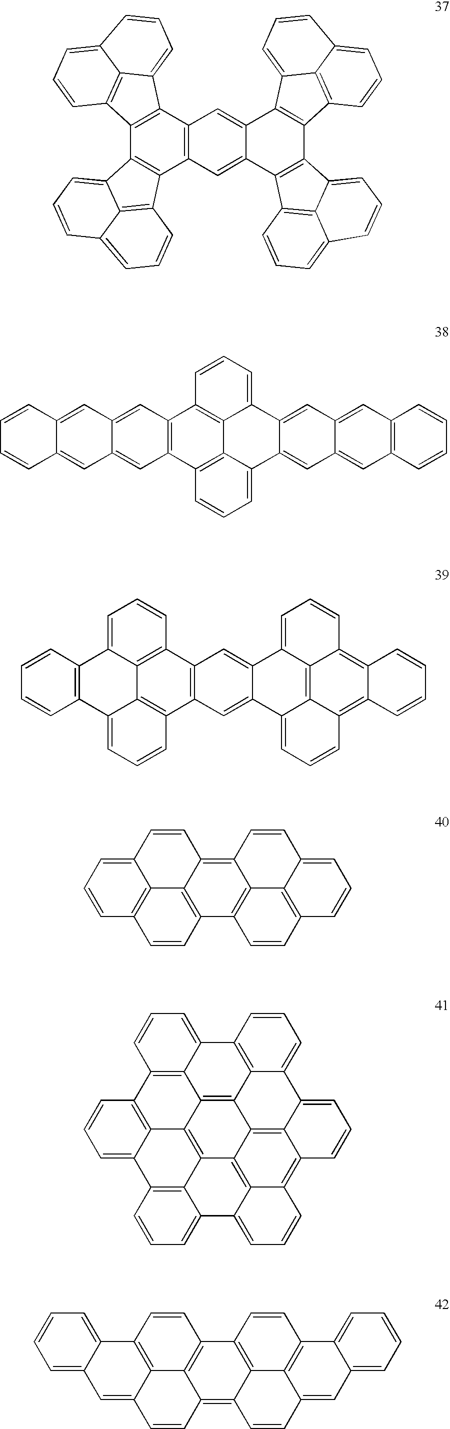

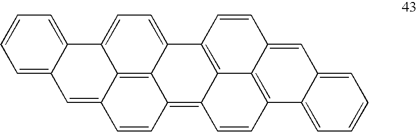

15. A film according to claim 11, wherein said organic compound comprises one or more coronene fragments, and wherein said organic compound comprising coronene fragments has a general structural formula from the group comprising structures 44-51:

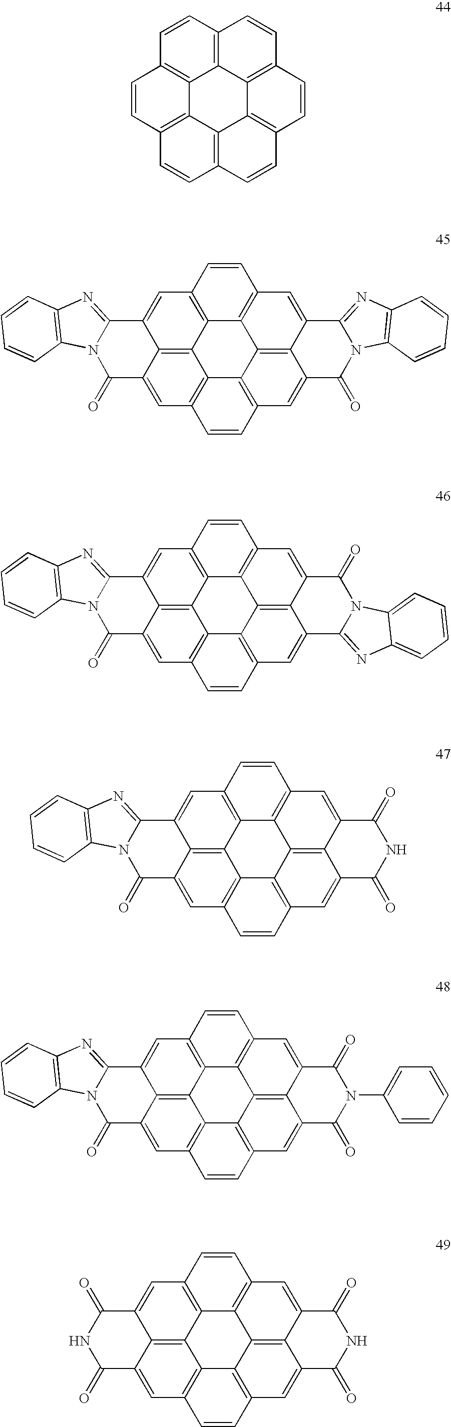

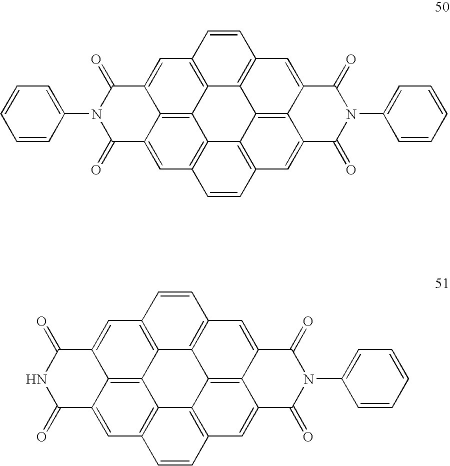

16. A device comprising at least one optically transparent and electrically conductive layer based on a ribtan material.

17. A device according to claim 16, wherein at least one of the optically transparent and electrically conductive layers is transparent in the UV, visible and near IR regions of optical spectrum.

18. A device according to claim 17, wherein the optically transparent and electrically conductive layer possesses polarizing properties in the visible spectral range.

19. A device according to claim 16, wherein the optically transparent and electrically conductive layer serves as electrode.

20. A device according to claim 19, wherein the device is selected from the list comprising an optoelectronic device, a touch screen, an electromagnetic shield, a sensor, and a liquid-crystal display.

21. A device according to claim 16, wherein at least one optically transparent and electrically conductive layer has an optical transparency of at least 80% for 550 nm light and a resistivity of less than 0.002-0.029 Ohm·cm.

22. A device according to claim 16, wherein the ribtan material is prepared using a solution comprising at least one π-conjugated organic compound of the general structural formula I or a combination of organic compounds of the general structural formula I: 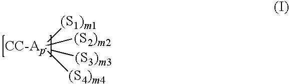
where CC is a predominantly planar carbon-conjugated core;
A is an hetero-atomic group;
p is 0, 1, 2, 3, 4, 5, 6, 7, or 8;
S₁, S₂, S₃, and S₄ are substituents;
m1, m2, m3 and m4 are 0, 1, 2, 3, 4, 5, 6, 7, or 8; and
sum (m1+m2+m3+m4) is 1, 2, 3, 4, 5, 6, 7, 8, 9 or 10.

23. A device according to claim 22, wherein said organic compound comprises one or more rylene fragments, and wherein said organic compound comprising rylene fragments has a general structural formula from the group comprising structures 1-23:

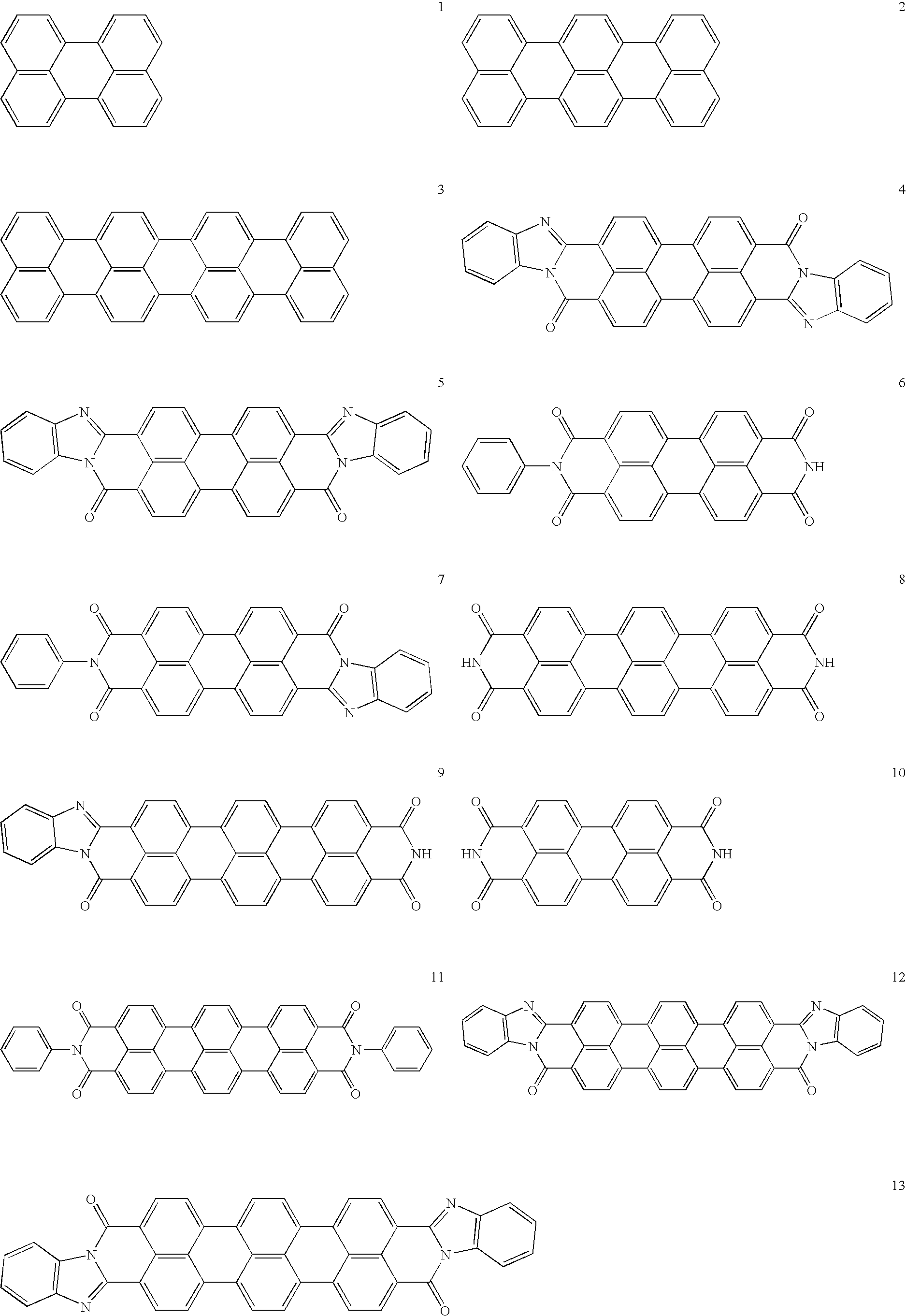

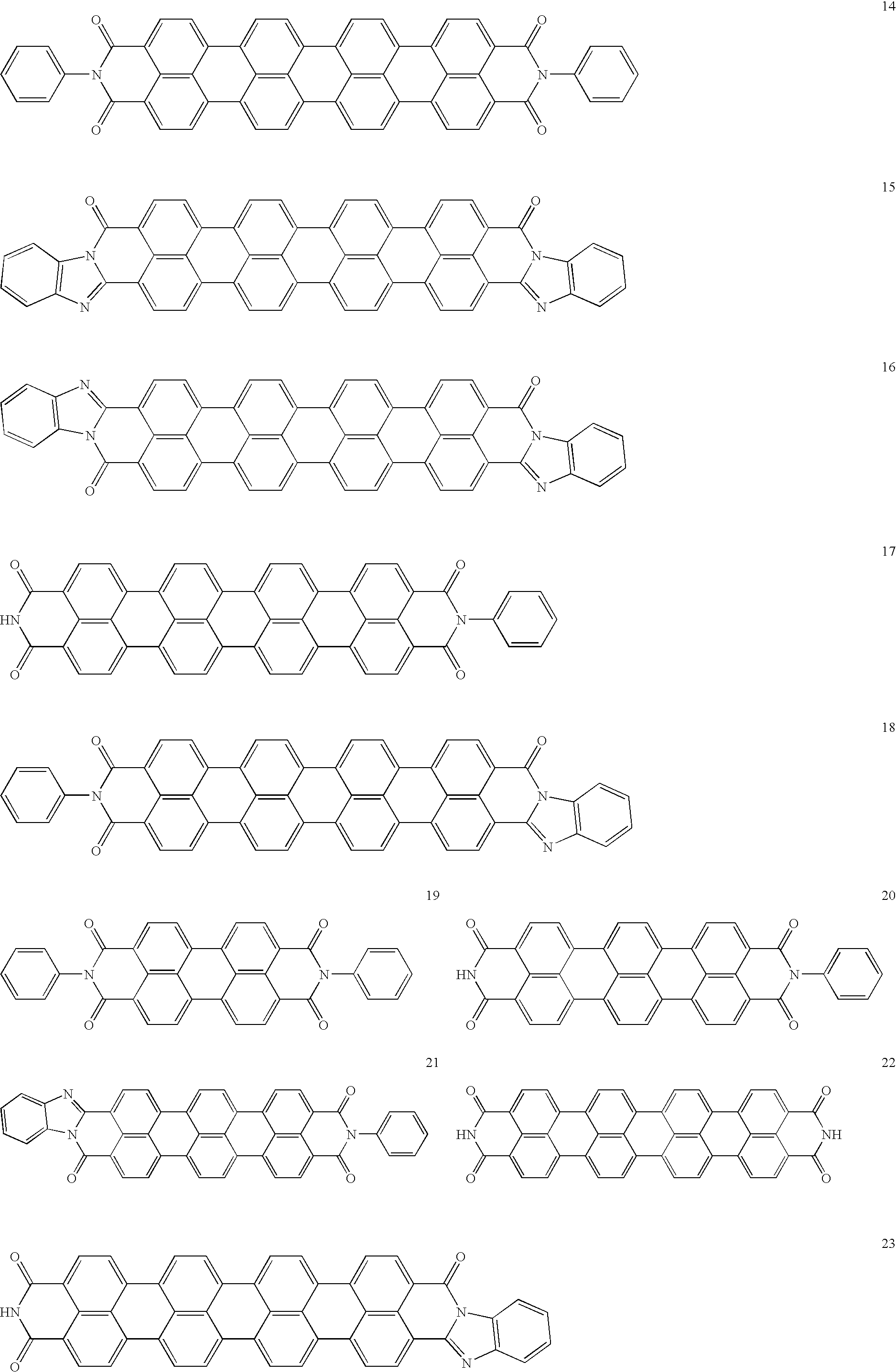

24. A device according to claim 22, wherein said organic compound comprises one or more anthrone fragments, and wherein said organic compound comprising anthrone fragments has a general structural formula from the group comprising structures 24-31:

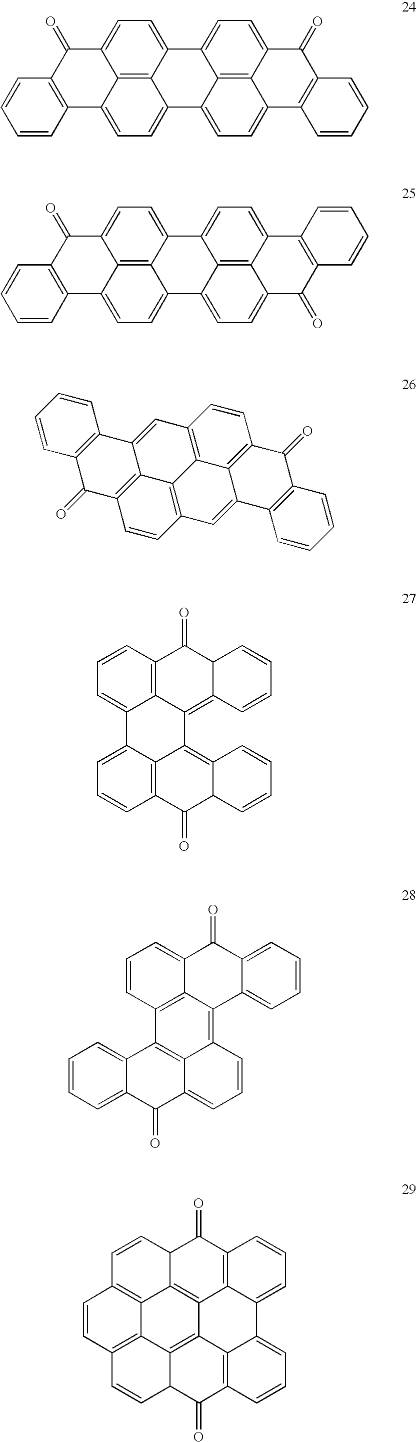

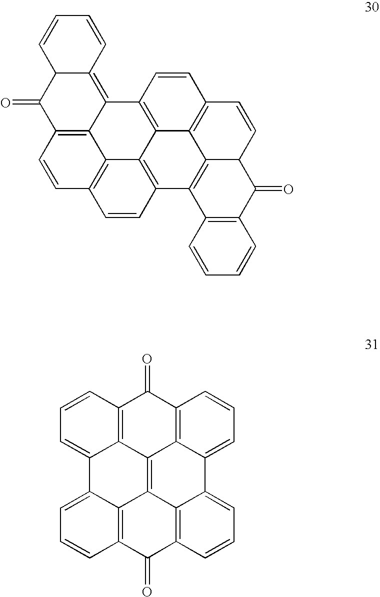

25. A device according to claim 22, wherein said organic compound comprises fused polycyclic hydrocarbons, and wherein said organic compound comprising fused polycyclic hydrocarbons is selected from the list comprising truxene, decacyclene, antanthrene, hexabenzotriphenylene, 1.2,3.4,5.6,7,8-tetra-(peri-naphthylene)-anthracene, dibenzoctacene, tetrabenzoheptacene, peropyrene, hexabenzocoronene, violanthrene, isoviolanthrene and has a general structural formula selected from the group comprising structures 32-43.

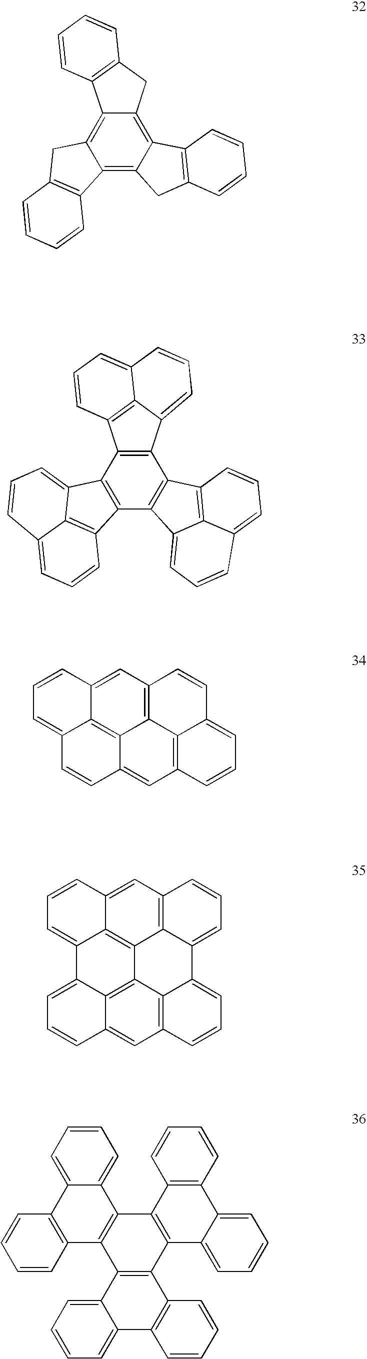

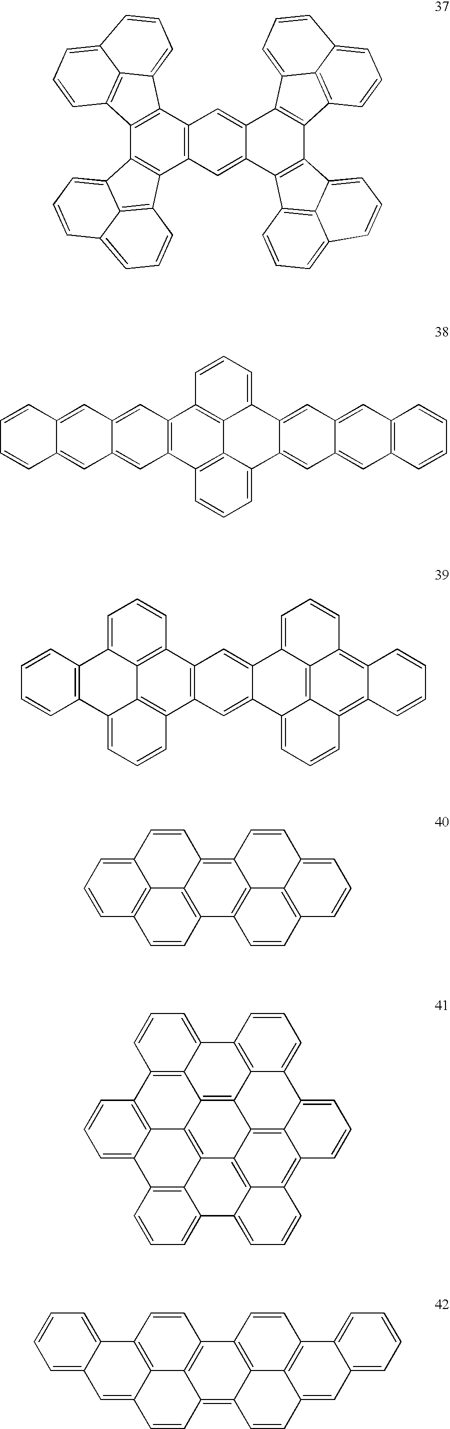

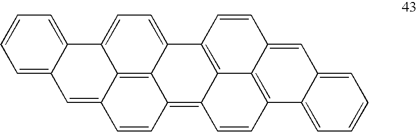

26. A device according to claim 22, wherein said organic compound comprises one or more coronene fragments, and wherein said organic compound comprising coronene fragments has a general structural formula from the group comprising structures 44-51:

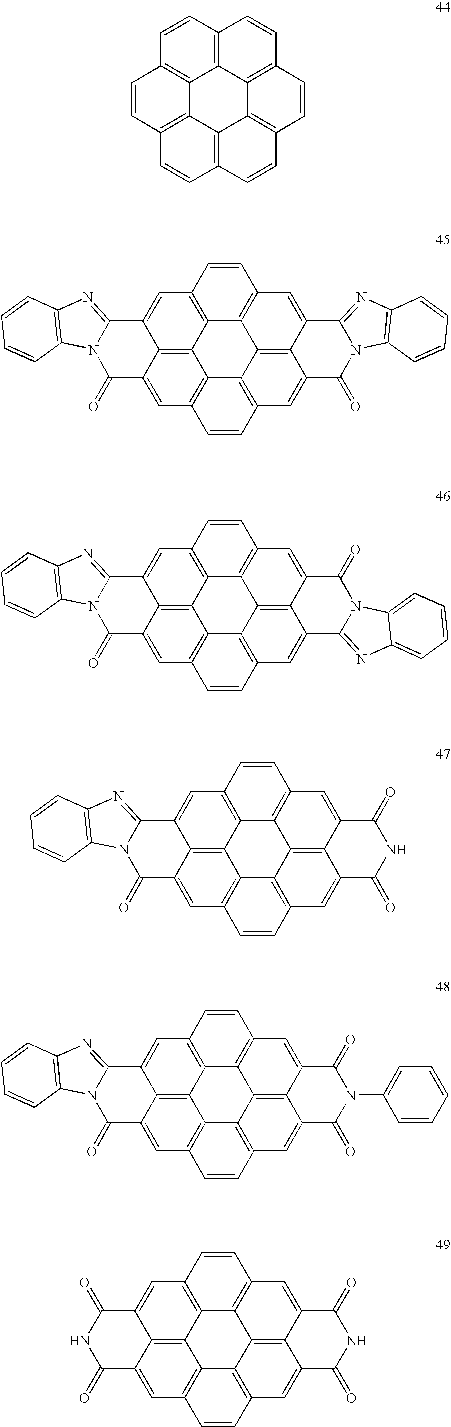

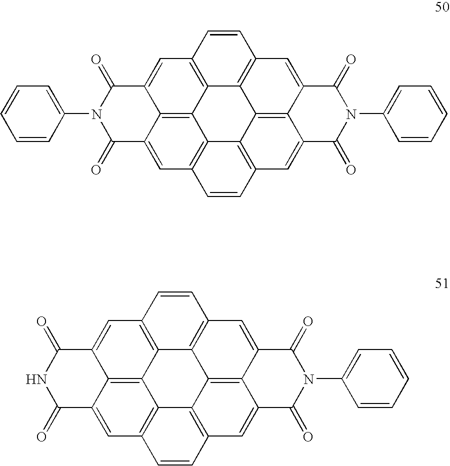

27. A method of producing at least one ribtan layer on a substrate, which comprises the following steps:
(a) application of a solution of at least one π-conjugated organic compound of the general structural formula I or a combination of organic compounds of the general structural formula I on a substrate: 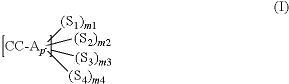
where CC is a predominantly planar carbon-conjugated core;
A is a hetero-atomic group;
p is 0, 1, 2, 3, 4, 5, 6, 7, or 8;
S₁, S₂, S₃, and S₄ are substituents,
m1, m2, m3 and m4 are 0, 1, 2, 3, 4, 5, 6, 7, or 8; and
sum (m1+m2+m3+m4) is 1, 2, 3, 4, 5, 6, 7, 8, 9 or 10;
(b) drying with formation of a solid precursor layer, and
(c) formation of a ribtan layer,
wherein said formation step is characterized by a level of vacuum, composition and pressure of ambient gas,
wherein at least one said graphene-like structure possesses conductivity and is predominantly continuous within the entire ribtan layer, and
wherein thickness of the ribtan layer is in the range from approximately 1 nm to 1000 nm.

28. A method of producing a ribtan layer on a substrate, which comprises the following steps:
(a) preparation of a solution of one π-conjugated organic compound of a general structural formula II or a combination of organic compounds of the general structural formula II capable of forming supramolecules: 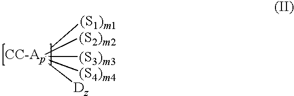
where CC is a predominantly planar carbon-conjugated core,
A is an hetero-atomic group, p is 0, 1, 2, 3, 4, 5, 6, 7, or 8,
S₁, S₂, S₃, S₄ and D are substituents,
where S₁, S₂, S₃, and S₄ are substituents providing solubility of the organic compound in a suitable solvent,
D is a substituent which produces reaction centers selected from the list comprising free radicals and benzyne fragments on the predominantly planar carbon-conjugated cores after a subsequent elimination of this substituent during a step (e),
m1, m2, m3 and m4 are 0, 1, 2, 3, 4, 5, 6, 7, or 8,
sum (m1+m2+m3+m4) is 1, 2, 3, 4, 5, 6, 7, 8, 9 or 10, and
z is 0, 1, 2, 3 or 4;
(b) deposition of a layer of the solution on the substrate
(c) alignment action upon the solution in order to ensure preferred alignment of the supramolecules;
(d) drying with formation of a solid precursor layer; and
(e) application of an external action upon the solid precursor layer stimulating low-temperature carbonization and formation of the ribtan layer.
